(19) United States
(12) Patent Application Publication
(10) Pub. No.: US 2011/0008415 A1
(43) Pub. Date: Jan. 13, 2011

(54) NOVEL LIPOPHILIC COMPOSITIONS AND USES THEREOF

(75) Inventors: Jean-Claude Clement, Kranidi (GR); Harivony Pichon, Saint Denis de l'Hotel (FR); Patrick Midoux, Saint Denis de l'Hotel (FR); Jean-Jacques Yaouanc, Locmaria-Plouzane (FR); Mathieu Mevel, Nantes (FR)

Correspondence Address: YOUNG & THOMPSON, 209 Madison Street, Suite 500, Alexandria, VA, 22314, US

(73) Assignees: UNIVERSITE DE BRETAGNE OCCIDENTALE, Brest Cedex 3 (FR); CENTRE NATIONAL DE LA RECHERCHE SCIENTIFIQUE, Paris Cedex 16 (FR)

(21) Appl. No.: 12/680,344

(22) PCT Filed: Sep. 25, 2008
(86) PCT No.: PCT/FR08/51709

§ 371 Date: May 3, 2010

(30) Foreign Application Priority Data
Sep. 28, 2007 (FR) 07 57955

(51) Int. Cl. A61K 9/127; C12N 15/63; C12N 5/071; A61K 47/18; A61K 31/7088; C07F 9/06; C07D 233/61; A61P 43/00

(52) U.S. Cl. 424/450; 435/320.1; 435/325; 514/788; 435/366; 514/44 R; 558/199; 548/335.5

(57) ABSTRACT

New co-lipids for use in combination with lipophilic compounds as nucleic acid vectors, for preparing a nucleic acid vector composition, and methods for introducing in vitro or in vivo a nucleic acid of interest into host cells, including a step of contacting host cells these nucleic acid vector compositions. The nucleic acid vector compositions have the combination (i) of a nucleic acid, cationic, lipophilic vector with (ii) a co-lipid. These nucleic acid vector compositions, in some embodiments, are present in the form of unilamellar or multilamellar vesicles.

### FIELD OF THE INVENTION

The present invention relates to the field of compositions for use as non viral vectors for introducing nucleic acids of interest into a human host cell or a non human mammal host cell.

### PRIOR ART

In recent years, many authors focused on the development of non viral vectors intended to carry a DNA of interest through the cell membrane and to the cell nucleus, especially in the context of gene therapy methods.

It could be noted from the journal of A. D. MILLER called “Cationic liposomes for gene therapy” (Angewandte Chem. Int., Ed. Engl., 1998, Vol. 37: 1768-1785), which is a general review dealing with cationic lipids, that the cation positive charge is always carried by a nitrogen atom.

To be mentioned as lipophilic compounds used in the state of the art as non viral vectors are 1,2-dioleyl-3 trimethylammonium deoxyglycerol halides, commonly referred to as DOTAP, 1,2-dioleyl-3 trimethylammonium, commonly referred to as DOTMA and dimethylammonium ethyloxycarbonylcholesterol, commonly referred to as DC-chol.

Phosphonolipids have also been described, such as those described by G. Le Bolc'h et al., (Tetrahedron Lett., 1995, 36, 6681) and V. Floch et al (Eur. J. Med. Chem., 1998, 33, 12.), phosphonolipids in the form of a ammonium cation salt (V. Floch et al., Eur. J. Med. Chem., 1998/, Vol. 33: 923-934) or in the form of a phosphonium or arsonium cation salt (E. Guénin et al., Angew. Chem Int. Ed., 2000, Vol. 39(3); V. Floch et al., J. Med. Chem., 2000, Vol. 43 (24): 4617-4628).

Generally speaking, non viral cationic lipophilic vectors have a poor DNA transfection efficiency in cells and possess cytotoxic properties towards these cells.

However, some cationic lipophilic vectors are known from the state of the art, which present good transfection yields together with a reduced cytotoxicity.

In order to reduce the cytotoxicity of cationic lipophilic vectors, their use has been described in the state of the art in combination with neutral “co-lipids” such as dioleoyl phosphatidyl ethanolamine (DOPE) or cholesterol. However, using such co-lipids for thus reducing the cytotoxic properties of some cationic lipophilic vectors does also bring drawbacks. It is known, for example, that the co-lipid DOPE causes the ability of the resulting vector lipid composition to transfect or transform a host cell with a nucleic acid of interest to be reduced, due to the fact that DOPE induces the aggregation of the complexes lipophilic vectors/nucleic acids with the blood lipoproteins.

There is therefore a need in the state of the art for new lipophilic compositions for use as nucleic acid vectors of interest, which have a high transfection ability and a reduced cytotoxicity.

There is especially a need for publicly available improved co-lipids to be used together with cationic lipophilic vectors.

There is also in the state of the art a need for non viral vectors carrying more efficiently a nucleic acid through the cell membrane, to the cell nucleus, so as to obtain transfection yields higher than those observed with known non viral vectors.

### SUMMARY OF THE INVENTION

First, there are provided new co-lipids according to the present invention, for use in combination with lipophilic compounds as nucleic acid vectors, for preparing a nucleic acid vector composition.

The present invention also provides nucleic acid vector compositions comprising the combination (i) of a nucleic acid, cationic, lipophilic vector with (ii) a co-lipid. These nucleic acid vector compositions in some embodiments present in the form of unilamellar or multilamellar vesicles.

The invention further relates to methods for introducing in vitro or in vivo a nucleic acid of interest into host cells, comprising a step of contacting said host cells with a nucleic acid vector composition such as defined hereabove.

The present invention also relates to complexes between a nucleic acid of interest and a nucleic acid vector composition such as defined hereabove.

Lastly the present invention relates to new cationic lipophilic compounds as nucleic acid vectors and to their applications.

### DESCRIPTION OF THE FIGURES

FIGS. 1 to 6 illustrate various synthesis schemes for preparing the lipophilic compounds of formulas (I) and (XI).

FIG. 1 shows the synthesis scheme for lipophosphoramidate compounds having a polar head composed of an amino acid derivative. FIG. 2 shows the synthesis scheme for lysine methyl ester-lipophosphoramidate 4 starting from compound 3d. FIG. 3 shows the synthesis scheme for compounds 5 and 6. FIG. 4 shows the synthesis scheme for an imidazole ring-containing lipophosphoramidate 7b and for an imidazolium ring-containing lipophosphoramidate 8b. FIG. 5 shows the synthesis scheme for compound 8a. FIG. 6 shows the synthesis scheme for compound 9.

FIG. 7: Expression kinetics of the transgene in rat's damaged tendons. 20 μg of vectorized or not vectorized pNFCMV-luc (40 μL of the end volume) are injected. The luciferase activity is evaluated 1 or 3 or 6 days following the transfection. The results do correspond to averages and standard deviations for 3 independent experiments in triplicate. The luciferase activity in the untreated collateral tendon was deducted for each treated rat. On the abscissa: time following the transfection, expressed as the number of days; bars from the left to the right: DNA with no vector, DNA complexed with a composition comprising the combination of compounds 8a and 9, DNA complexed with Jet-PEI polymer. Ordinates: the results for the transfection efficiency corresponding to the luciferase activity found in samples of transfected cells in culture, expressed in TRLU units (“Total Relative Light Units”), as described in the section “Materials and methods” in the examples.

FIG. 8: Toxicity assessment. Cultured tenocytes were transfected with 8a/9 and JetPEI. MTT test was carried out after 48 h. The toxicity percentage was established as compared to non transfected control cells. On the abscissa: sample type. Ordinates: cytotoxicity percentage.

FIG. 9: Achilles tendon histological analysis. The tendons were surgically damaged and transfected (or not) with plasmid pBlast hB-PDGF encoding growth factor PDGF vectorized with 8a/9. The tendons were then collected at various days for histological analysis: longitudinal sections (thickness 4 μm) followed by HES staining.

### Magnification 20×:

9A-1 and 9B-1: Control undamaged tendon.
9C-1: Damaged tendon, at day 3, not treated.
9D-1: Damaged tendon, at day 3, transfected with cDNA of PDGF vectorized with 8a/9.
9E-1: Damaged tendon, at day 6, not treated.
9F-1: Damaged tendon, at day 6, transfected with pBlast hB-PDGF vectorized with 8a/9.

### Magnification 40×:

9A-2: Control undamaged tendon.
9B-2: Damaged tendon, at day 3, not treated.
9C-2: Damaged tendon, at day 6, not treated.
9D-2: Damaged tendon, at day 6, transfected with pBlast hB-PDGF vectorized with 8a/9.

### DESCRIPTION OF THE INVENTION

The applicants did synthesize new lipophilic compounds for use as co-lipids in non viral nucleic acid vector compositions.

More precisely, the applicants developed lipophilic compounds for use as co-lipids, which represent compounds of the lipophosphoramidate family, which are non ionized at physiological pH, and which become cationic at acidic pH. A “physiological pH” is intended to mean a pH value ranging from 7 to 7.6, and typically a pH of 7.4. As used herein, an “acidic pH” is intended to mean a pH value of less than 7.

It is an object of the present invention to provide the use of a lipophilic compound of following formula (I):

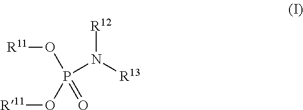

wherein (i) R¹¹ and R′¹¹ each represent independently from each other, an alkyl chain having from 10 to 24 carbon atoms, a monoalkenyl or a polyalkenyl chain having from 10 to 24 carbon atoms, the polyalkenyl chain having from 2 to 4 double bonds, or a monoalkynyl or a polyalkynyl chain having from 10 to 24 carbon atoms, the polyalkynyl chain having from 2 to 4 triple bonds;
(ii) R¹² is a hydrogen atom or an alkyl chain having from 1 to 4 carbon atoms;
(iii) R¹³ is selected from:

(iii-1) a group of formula

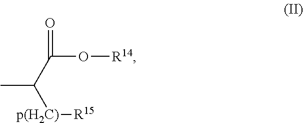

wherein

R¹⁴ is an alkyl group having from 1 to 4 carbon atoms;

p is an integer equal to 1, 2, 3 or 4; and

R¹⁵ is the following group:

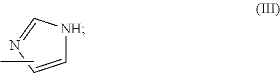

or

(iii-2) a group of formula —(CH₂)_{q}—R¹⁶, wherein

q is an integer equal to 1, 2, 3 or 4;

R¹⁶ is the following group:

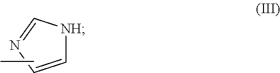

as a co-lipid for preparing a nucleic acid non viral vector composition.

It has been shown according to the invention that a lipophilic compound of formula (I) hereabove has the ability to increase the transfection properties of cationic lipophilic vectors, including non viral, cationic lipophilic vectors of the cationic phosphoramide type.

It has also been shown according to the invention that a lipophilic compound of formula (I) hereabove is not cytotoxic. In addition, a lipophilic compound of formula (I) hereabove, when used as a co-lipid in combination with a lipophilic cationic, vector compound, enables to prepare nucleic acid vector lipophilic compositions with a reduced cytotoxicity.

It has been shown in particular that with lipophilic compounds of formula (I) used as co-lipids in lipophilic, vector compositions, the end composition has very low cytotoxicity properties, as compared to the cytotoxicity properties of lipophilic, vector compositions which comprise traditional co-lipids such as DOPE (L-α-dioleolyl-phosphatidyl ethanolamine).

Thus, new lipophilic compounds of formula (I) hereabove enable to prepare vector compositions possessing combined properties as (i) a high ability to transfect nucleic acids of interest in host cells and (ii) a reduced cytotoxicity and even, in some embodiments, a near absence of cytotoxic properties.

In a preferred embodiment of use of a lipophilic compound of formula (I) hereabove, said co-lipid compound is combined with a cationic, lipophilic compound, that is able to form a complex with a nucleic acid.

It is also an object of the present invention to provide a lipophilic composition comprising the combination of two lipophilic compounds, respectively:
a) a first cationic, lipophilic compound, that is able to form a complex with a nucleic acid; and
b) a second lipophilic compound of following formula (I):

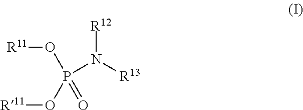

wherein (i) R¹¹ and R′¹¹ each represent independently from each other, an alkyl chain having from 10 to 24 carbon atoms, a monoalkenyl or a polyalkenyl chain having from 10 to 24 carbon atoms, the polyalkenyl chain having from 2 to 4 double bonds, or a monoalkynyl or a polyalkynyl chain having from 10 to 24 carbon atoms, the polyalkynyl chain having from 2 to 4 triple bonds;
   (ii) R¹² is a hydrogen atom or an alkyl chain having from 1 to 4 carbon atoms;
   (iii) R¹³ is selected from:
      (iii-1) a group of formula

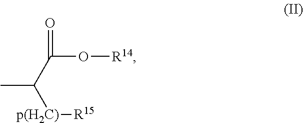

wherein
R¹⁴ is an alkyl group having from 1 to 4 carbon atoms;
p is an integer equal to 1, 2, 3 or 4; and
R¹⁵ is the following group:

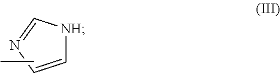

or
(iii-2) a group of formula —(CH₂)_{q}—R¹⁶, wherein
q is an integer equal to 1, 2, 3 or 4;
R¹⁶ is the following group:

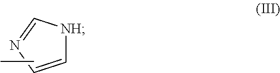

As used herein, an “alkyl” is intended to mean a linear or branched, aliphatic hydrocarbon group. The alkyl chain may be substituted, on one or more of the carbon atoms thereof, by one or more groups selected from methyl, hydroxy, alcoxy and alkylthio groups. Preferably, a carbon atom in the hydrocarbon chain comprises at most only one substituent, but said carbon atom may comprise two substituents. In the alkyl chain, all the carbon atoms may comprise at least one substituent amongst the hereabove mentioned substituents.

For the R¹¹ and R′¹¹ groups, alkyl, monoalkenyl, polyalkenyl, monoalkynyl and polyalkynyl chains preferably have from 14 to 20 carbon atoms. Preferred R¹¹ and R′¹¹ groups especially comprise alkyl, monoalkenyl, polyalkenyl, monoalkynyl and polyalkynyl chains with 16 or 18 carbon atoms.

In some embodiments of lipophilic compounds of formula (I), wherein the R¹¹and/or R′¹¹ groups represent(s) an alkyl chain, said alkyl chain is substituted by at least one methyl group, for example by 2 to 8 methyl groups. In some embodiments, the R¹¹and/or R′¹¹ groups represent(s) a phytanyl group, that is to say an alkyl chain having 16 carbon atoms from which four are monosubstituted by a methyl group, i.e. carbon atoms at positions 3, 7, 11 and 15, respectively.

As used herein, a “monoalkenyl” is intended to mean an alkyl group comprising a carbon-carbon double bond, which may be located anywhere within the hydrocarbon chain.

As used herein, a “polyalkenyl” is intended to mean an alkyl group comprising from two to four carbon-carbon double bonds in the hydrocarbon chain, which may be located anywhere within the hydrocarbon chain in “malonic” relative positions.

As used herein, a “monoalkynyl” is intended to mean an alkyl group comprising a carbon-carbon triple bond, which may be located anywhere within the hydrocarbon chain.

As used herein, a “polyalkynyl” is intended to mean an alkyl group comprising from two to four carbon-carbon triple bonds in the hydrocarbon chain, which may be located anywhere within the hydrocarbon chain in “malonic” relative positions.

According to the invention, a complex between a cationic, lipophilic compound and a nucleic acid does mean that said nucleic acid is bound to the cationic, lipophilic compound through non covalent bonds, due to the nucleic acid ability, whether of sRNA or sDNA, to associate with no covalent bond to positively charged substances.

As used herein, a “cationic, lipophilic compound” is intended to mean a compound comprising (i) at least one lipophilic hydrocarbon chain and (ii) at least one chemical group which is positively charged at physiological pH, said compound being able to form a complex with a nucleic acid.

The lipophilic composition as defined hereabove represents the lipophilic, non viral vector composition of the invention. This lipophilic composition forms a complex with nucleic acids of interest. As previously mentioned, the hereabove lipophilic composition does possess outstanding properties for transfecting host cells with nucleic acids, associated with reduced cytotoxic properties.

A first family of compounds of formula (I) is that family of compounds of formula (I), wherein the R¹³ group is a group of formula (II), which is illustrated in the examples especially through the compound noted 3c.

In a first preferred embodiment of the compounds of formula (I), wherein the R¹³ group is a group of formula (II), the R¹² group represents a hydrogen atom.

In a second preferred embodiment of the compounds of formula (I), wherein the R¹³ group is a group of formula (II), the R¹⁴ group represents a methyl group.

In a third preferred embodiment of the compounds of formula (I), wherein the R¹³ group is a group of formula (II), p is 1, 3 or 4.

In a fourth preferred embodiment of the compounds of formula (I), wherein the R¹³ group is a group of formula (II), the R¹⁵ group is selected from the following groups:

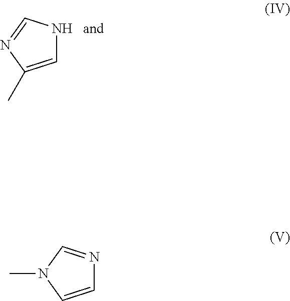

A second family of compounds of formula (I) is that family of compounds of formula (I), wherein the R¹³ group is a group of formula —(CH₂)_{q}—R¹⁶, which is illustrated in the examples especially through the compounds noted 7b and 9.

In a first preferred embodiment of the compounds of formula (I), wherein the R¹³ group is a group of formula —(CH₂)_{q}—R¹⁶, the R¹² group represents a hydrogen atom.

In a second preferred embodiment of the compounds of formula (I), wherein the R¹³ group is a group of formula —(CH₂)_{q}—R¹⁶, q is 2 or 3.

In a third preferred embodiment of the compounds of formula (I), wherein the R¹³ group is a group of formula —(CH₂)_{q}—R¹⁶, the R¹⁶ group is selected from the following groups:

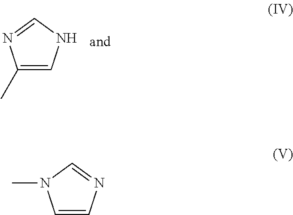

In a preferred embodiment of the compounds of formula (I), the R¹¹ and R′¹¹ groups independently from each other are selected from:
a tetradecyl group,
an oleyl group,
a phytanyl group,
C_{18:2} and C_{18:3} polyalkenyl groups, wherein the first number is the number of carbon atoms in the alkenyl chain and the second number is the number of double bonds in the alkenyl chain; and
a C_{18:1} monoalkenyl group, wherein the first number is the number of carbon atoms in the alkenyl chain and the second number is the number of double bonds in the alkenyl chain.

In another preferred embodiment of the compounds of formula (I), the R¹¹ and R′¹¹ groups are the same.

Most preferably, the R¹¹ and R′¹¹ groups each represent an oleyl group.

The lipophilic compounds of formula (I) of the invention consist in non ionized compounds when they are in a solution at a physiological, neutral pH (pH value 7.6). On the contrary, the lipophilic compounds of formula (I) consist in cationic ionized compounds when they are in a solution at an acidic pH of less than 7, typically at an acidic pH of less than 6.

Without wishing to be bound by any theory, the applicant thinks that the lipophilic compounds of formula (I) do possess good fusion properties with lipids, and especially with membrane lipids of cells or some intracellular vesicles, including endosomes. The cationic character in an acidic medium of the lipophilic compounds of formula (I) of the invention is such that it could contribute to destabilize the endosomal vesicles of the cell cytoplasm following an osmosis, as it is known that endosomal vesicles do act as proton pumps. Thus, after having been combined with cationic lipophilic vectors, the lipophilic compounds of formula (I) promote, as co-lipids, the cationization of the lipophilic nucleic acid vector compositions of the invention in the cell cytoplasm, and more particularly in the endosomal vesicles, which would favor the delivery of nucleic acids in the cytosol and would thus explain their yield increasing properties of cell transfection with the nucleic acids of interest.

In a lipophilic nucleic acid vector composition according to the invention, the cationic, lipophilic compound used in combination with the co-lipid of formula (I) may be any of known cationic, lipophilic compounds which can form a complex with a nucleic acid, either DNA or RNA. As a rule, a cationic, lipophilic compound as a nucleic acid vector comprises: (i) a lipophilic part, generally speaking one or more hydrocarbon chains having from 10 to 24 carbon atoms, saturated or mono- or poly-unsaturated, (ii) a cationic part which is positively charged in a solution at physiological pH and (iii) a linkage group (“linker”) which binds the lipophilic part to the cationic part, and which generally consists in an acyl bond or an ether bond.

For example, the cationic, lipophilic compound as a nucleic acid vector may be selected from 1,2-dioleyl-3 trimethylammonium deoxyglycerol (DOTAP), 1,2-dioleyl-3 trimethylammonium (DOTMA), dimethylammonium ethyloxycarbonylcholesterol (DC-chol), dimethyldioctadecyl ammonium bromide (DDAB), 1,2-dimyristoyl-3 trimethylammonium deoxyglycerol, 1,2-dipalmitoyl-3 trimethylammonium deoxyglycerol, 1,2-dioleyl-3 trimethylammonium deoxyglycerol, 1,2-distearoyl-3 trimethylammonium deoxyglycerol, N-[1-[2,3-bis(oleoyloxy)]propyl-1]-N,N,N-trimethylammonium chloride, dioctadecyl amidoglycylspermine (DOGS), 2,3-dioleoyloxy-N-(2(sperminecarboxamido)-ethyl)-N,N-dimethyl-1-propanaminium trifluoroacetate (DOSPA), 1,2-dioleoyl-sn-glycero-3-ethyl phosphocholine (DOEPC), 1,2-dilauroyl-sn-glycero-3-ethyl phosphocholine, 1,2-dipalmitoyl-sn-glycero-3-ethyl phosphocholine, 1,2-distearoyl-sn-glycero-3-ethyl phosphocholine, 1,2-palmitoyl-oleoyl-sn-glycero-3-ethyl phosphocholine, 1-[2-(9(Z)-octadecenoyloxy)ethyl]-2-(8(Z)-heptadecenyl 1-3-(2-hydroxyethyl)imidazolium chloride .(DOTIM), 1-[2-tetradecanoyloxy)ethyl]-2-tridecyl-3-(2-hydroxyethyl)imidazolium chloride (DPTIM), 1-[2-tetradecanoyloxy)ethyl]-2-tridecyl-3-(2-hydroxyethyl)imidazolium chloride, 1,2-dioleoyl-3-dimethyl-hydroxyethyl ammonium bromide (DOR1); 1,2-dioleyloxypropyl-3-dimethyl-hydroxyethyl ammonium bromide (DORIE); 1,2-dioleyloxypropyl-3-dimethyl-hydroxypropyl ammonium bromide (DORIE-HP); 1,2-dioleyloxypropyl-3-dimethyl-hydroxybutyl ammonium bromide (DORIE-HB); 1,2-dioleyloxypropyl-3-dimethyl-hydroxypentyl ammonium bromide (DORIE-HPe); 1,2-dimyristyloxypropyl-3-dimethyl-hydroxyethyl ammonium bromide (DMRIE); 1,2-dipalmityloxypropyl-3-dimethyl-hydroxyethyl ammonium bromide (DPRIE), 1,2-disteryloxypropyl-3-dimethyl-hydroxyethyl ammonium bromide (DSRIE); L-histidine-(N,N-di-n-hexadecylamine)ethylamide (lipid 1); L-histidine-(N,N-di-n-hexadecylamine-N-methyl)ethylamide; L-histidine-cholesteryl-ethylamide, alanine-cholesteryl-ethylamide, and bis(guanidinium)-tren-cholesterol (BGTC).

All of the hereabove mentioned cationic lipophilic compounds acting as nucleic acid vectors are commercially available products or the synthesis thereof is described in the technical literature.

In a preferred embodiment of a lipophilic nucleic acid vector composition according to the invention, the cationic, lipophilic compound with which the lipophilic compound of formula (I) is combined, is a lipophilic compound of following formula (XI):

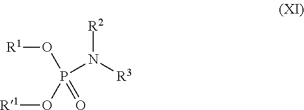

wherein:
(i) R¹ and R′¹ each represent independently from each other, an alkyl chain having from 10 to 24 carbon atoms, a monoalkenyl or a polyalkenyl chain having from 10 to 24 carbon atoms, the polyalkenyl chain having from 2 to 4 double bonds, or a monoalkynyl or a polyalkynyl chain having from 10 to 24 carbon atoms, the polyalkynyl chain having from 2 to 4 triple bonds;
(ii) R² is a hydrogen atom or an alkyl chain having from 1 to 4 carbon atoms;
(iii) R³ is selected from:
   (iii-1) a group of formula

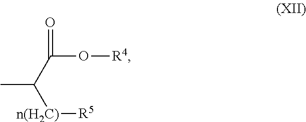

wherein
R⁴ is an alkyl group having from 1 to 4 carbon atoms;
n is an integer equal to 1, 2, 3 or 4; and
R⁵ is a group selected from:

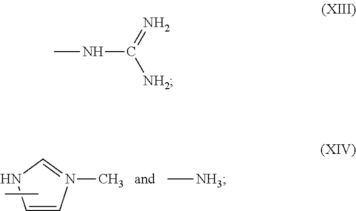

or
(iii-2) a group of formula —(CH₂)ₒ—R⁶, wherein
o is an integer equal to 1, 2, 3 or 4;
R⁶ is a group selected from:

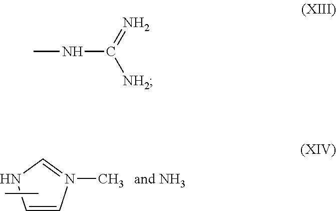

It has been shown according to the invention that the cationic, lipophilic compounds of formula (XI) defined hereabove represent outstanding lipophilic compounds for use as nucleic acid non viral vectors. The cationic, lipophilic compounds of formula (XI) comprise (i) a lipophilic part composed of two hydrocarbon chains having from 10 to 24 carbon atoms, (ii) a cationic part which is positively charged in a solution at physiological, neutral pH, preferably selected from (ii-a) an amino acid side chain that is positively charged at a physiological pH and (ii-b) an imidazolium group and (iii) a linkage group (“linker”) of the phosphoramidate type.

It has been especially shown according to the present invention that the compounds of formula (XI) hereabove, and even more specifically the compounds of formula (XI), wherein the R³ group is a group of formula (XII), have reduced cytotoxic properties, lower than the cytotoxic properties of the DNA vector, cationic lipids that are traditionally used in the state of the art, such as DOTAP and DOTMA.

In the cationic, lipophilic compounds of formula (XI) hereabove, the R³ group is protonated at a physiological pH, which makes the compounds of formula (XI) capable of complexing with nucleic acids.

Especially, in the cationic, lipophilic compounds of formula (XI), wherein the R³ group represents a group of formula (XII), said group of formula (XII) is that part of an amino acid comprising the carboxyl group, which is esterified, as well as the basic side chain, which is positively charged at a physiological pH.

The lipophilic compounds of formula (XI), wherein the R³ group represents a group of formula (XII) are new compounds.

A first family of compounds of formula (XI) is that family of compounds of formula (XI), wherein the R³ group represents a group of formula (XII), which is illustrated in the examples especially through the compounds noted 3a, 3b and 4.

In a first preferred embodiment of the compounds of formula (XI), wherein the R³ group is a group of formula (XII), the R² group represents a hydrogen atom.

In a second preferred embodiment of the compounds of formula (XI), wherein the R³ group is a group of formula (XII), the R⁴ group represents a methyl group.

In a third preferred embodiment of the compounds of formula (XI), wherein the R³ group is a group of formula (XII), n is 1, 3 or 4.

In a fourth preferred embodiment of the compounds of formula (XI), wherein the R³ group is a group of formula (XII), the R⁵ group is the following group:

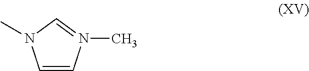

A second family of compounds of formula (XI) is that family of compounds of formula (XI), wherein the R³ group is a group of formula —(CH₂)ₒ—R⁶, which is illustrated in the examples especially through the compounds noted 6a, 6b, 8a and 8b.

In a first preferred embodiment of the compounds of formula (XI), wherein the R³ group is a group of formula —(CH₂)ₒ—R⁶, the R² group represents a hydrogen atom.

In a second preferred embodiment of the compounds of formula (XI), wherein the R³ group is a group of formula —(CH₂)ₒ—R⁶, o is 2 or 3.

In a third preferred embodiment of the compounds of formula (XI), wherein the R³ group is a group of formula —(CH₂)ₒ—R⁶, the R⁶ group is the following group:

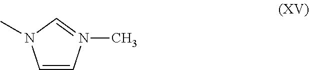

In a preferred embodiment of the compounds of formula (XI), the R¹ and R′¹ groups independently from each other are selected from:
a tetradecyl group,
an oleyl group,
a phytanyl group,
C_{18:2} and C_{18:3} polyalkenyl groups, wherein the first number is the number of carbon atoms in the alkenyl chain and the second number is the number of double bonds in the alkenyl chain; and
a C_{18:1} monoalkenyl group, wherein the first number is the number of carbon atoms in the alkenyl chain and the second number is the number of double bonds in the alkenyl chain.

In another preferred embodiment of the compounds of formula (XI), the R¹ and R′¹ groups are the same.

Most preferably, the R¹ and R′¹ groups each represent an oleyl group.

In a preferred embodiment of a lipophilic nucleic acid vector composition according to the invention, the first cationic, lipophilic compound of formula (XI) is selected from the following lipophilic compounds:
arginine methyl ester dioleoyl-N-phosphoramidate noted 3a,
homoarginine methyl ester dioleoyl-N-phosphoramidate noted 3b,
lysine methyl ester dioleoyl-N-phosphoramidate noted 4,
2-ethylguanidinium dioleoyl-N-phosphoramidate noted 6a,
4-butylguanidinium dioleoyl-N-phosphoramidate noted 6b,
2-ethyl(N-methyl)imidazolium dioleoyl-N-phosphoramidate noted 8a, and
3-propyl(N-methyl)imidazolium dioleoyl-N-phosphoramidate noted 8b.

In another preferred embodiment of a lipophilic nucleic acid vector composition according to the invention, the second lipophilic compound of formula (I) is selected from the following lipophilic compounds:
histidine methyl ester dioleoyl-N- phosphoramidate noted 3c,
3-propylimidazole dioleoyl-N- phosphoramidate noted 7b, and
histamine dioleoyl-N-phosphoramidate noted 9.

The invention includes any possible combination of the first and second lipophilic compounds that are teached in the present specification.

A particularly preferred lipophilic composition of the invention is that composition comprising the first lipophilic compound 8a and the second lipophilic compound 9.

Preferably, a lipophilic composition such as previously defined in the present specification is in the form of lipid vesicles, which may also be referred to as liposomes.

A lipophilic composition of the invention is preferably prepared in the form of lipid vesicles, which are then contacted with a nucleic acid of interest so as to form a complex between said nucleic acid and the thus prepared lipid vesicles.

It is another object of the invention to provide lipid vesicles substantially made of a lipophilic nucleic acid vector composition such as previously defined in this specification. As used herein, “substantially made of” is intended to mean that the lipid vesicles do comprise at least 90% by weight of the lipophilic composition of the invention, as compared to the lipid vesicle total weight.

The lipid vesicles are prepared from a mixture comprising:
a) a first cationic, lipophilic compound, that is able to form a complex with a nucleic acid; and
b) a second lipophilic compound of following formula (I):

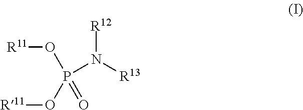

wherein
(i) R¹¹ and R′¹¹ each represent independently from each other, an alkyl chain having from 10 to 24 carbon atoms, a monoalkenyl or a polyalkenyl chain having from 10 to 24 carbon atoms, the polyalkenyl chain having from 2 to 4 double bonds, or a monoalkynyl or a polyalkynyl chain having from 10 to 24 carbon atoms, the polyalkynyl chain having from 2 to 4 triple bonds;
(ii) R¹² is a hydrogen atom or an alkyl chain having from 1 to 4 carbon atoms;
(iii) R¹³ is selected from:
   (iii-1) a group of formula

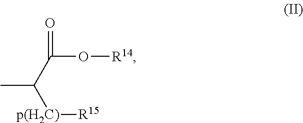

wherein
R¹⁴ is an alkyl group having from 1 to 4 carbon atoms;
p is an integer equal to 1, 2, 3 or 4; and
R¹⁵ is the following group:

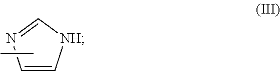

or
(iii-2) a group of formula —(CH₂)_{q}—R¹⁶, wherein
q is an integer equal to 1, 2, 3 or 4;
R¹⁶ is the following group:

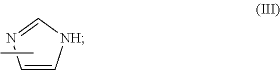

As already mentioned in the present specification, the first lipophilic compound, which consists in a cationic, lipophilic compound, may be any known cationic, lipophilic compound, which can form a complex with a nucleic acid, either DNA or RNA.

For the second lipophilic compound of formula (I), the meanings for the R¹¹, R′¹¹, R¹², R¹³, R¹⁴, R¹⁵ and R¹⁶ groups are such as previously defined in the present specification.

Included in the present invention are therefore lipid vesicles comprising a first cationic, lipophilic compound such as defined hereabove and a second lipophilic compound which consists in a lipophilic compound of formula (I) and which is used as a co-lipid.

Also included in the present invention are lipid vesicles formed exclusively of a lipophilic composition of the invention such as defined hereabove.

In a lipophilic composition of the invention, the first cationic, lipophilic compound and the second neutral lipophilic compound of formula (I) are used in a cationic compound to neutral compound molar ratio ranging from 1:1 to 3:2.

In the particular embodiment of the hereabove lipid vesicles, wherein said vesicles comprise less than 100% by weight of a lipophilic composition of the invention, said vesicles comprise up to 10% by weight of one or more other additional substances. Preferably, the additional substance(s) are one or more additional lipophilic compounds, which are generally lipophilic compounds known from the state of the art, such as DOPE, DOPC, or cholesterol, which are used as co-lipids.

Such lipid vesicles comprise at most four, and preferably at most two, distinct cationic, lipophilic compounds of the invention. Also, such lipid vesicles comprise at most four, and preferably at most two, distinct, neutral lipophilic compounds of the invention.

The lipid vesicles of the invention are prepared according to any method known from the person skilled in the art, especially according to methods for making liposomes, including those methods described in the examples. For example, the lipid vesicles of the invention may be prepared by dissolving beforehand one or more lipophilic compound(s) in an organic solvent, such as ethanol, then by injecting the resulting solution into an aqueous medium, for example pyrogen-free, distilled water or a physiologically compatible, saline solution, thereafter the lipid vesicles are formed by processing the thus obtained solution with ultrasounds, according to methods that are well known from the person skilled in the art.

Once the lipophilic compounds are dispersed in water or in an aqueous solution, or even in any hydrophilic solution, said lipophilic compounds produce membranes that are formed of lipid bi-layers also called lamellas. Lamellas are composed of two mono-layers of lipophilic compounds, their hydrophobic surface facing each other, and their hydrophilic surface contacting the aqueous medium, that is to say contacting both the outer environment aqueous medium and the aqueous medium contained in the inner space of the vesicles.

In some embodiments, the vesicles of the invention consist in unilamellar vesicles which comprise a single lipid bi-layer and have generally a diameter ranging from 100 to 200 nanometers.

In some other embodiments, the vesicles of the invention consist in multilamellar vesicles comprising traditionally from 2 to a few hundred concentric lipid bi-layers alternating with aqueous medium layers, which have generally a diameter ranging from 100 to 200 nanometers.

The lipophilic compounds in an organic solution may be stored in the long term, for example at a temperature ranging from 4 to 8° C. An extemporaneous preparation of the vesicles is preferably prepared, at most a couple of hours, for example at most 4 hours, prior to being contacted or incubated with a nucleic acid of interest.

Advantageously, the nucleic acid of interest which should be introduced into a host cell encodes a protein or a peptide. The protein may be any protein useful for implementing a gene therapy method, preferably a somatic gene therapy, and includes, without being limited thereto, cytokines, structural proteins, hormones, antigens, immunogens, receptors, etc.

In a second advantageous embodiment, the nucleic acid of interest encodes a sense or antisense polynucleotide, or an interfering RNA hybridizing with a target nucleic acid encoding a protein which expression inhibition in a host cell is sought for.

In another advantageous embodiment, the nucleic acid of interest consists in a messenger RNA encoding a protein of interest.

In a further advantageous embodiment, the nucleic acid of interest consists in a recombinant vector, preferably a recombinant expression vector, into which the nucleic acid of interest has been inserted, the coding sequence of which is placed under the control of regulating sequences, especially promoter or activating sequences (“enhancer”), that are required for expressing said nucleic acid of interest in the transfected host cell.

According to the invention, the nucleic acid of interest, either linear or circular, single strand, or double strand, is first complexed with a lipophilic composition according to the invention, which can already present in the form of lipid vesicles, prior to being introduced, as a complex, into the host cell.

However, the complexes are preferably formed by incubating the nucleic acid with the lipid vesicles as defined hereabove.

Under some circumstances, the complexes may be formed by incubating the nucleic acid of interest with a lipophilic composition of the invention, the thus formed complexes being subsequently used for transfecting the host cells. In an alternative, unilamellar or multilamellar lipid vesicles are formed from the nucleic acid/ lipophilic compound complexes prepared beforehand, thereafter the vesicles are used for transfecting the host cells.

It is also an object of the invention to provide the use of a lipophilic composition or of a lipid vesicle such as defined hereabove, in order to introduce, in vitro or in vivo, a nucleic acid into a host cell or into a host organism.

The invention further relates to a method for introducing, in vitro or in vivo, a nucleic acid into a host cell or a host organism, characterized in that it comprises the following steps of:
a) contacting said nucleic acid with a lipophilic composition or with a lipid vesicle such as defined hereabove, so as to obtain a complex between said nucleic acid, on the one hand, and said composition or said lipid vesicle, on the other hand;
b) incubating the host cell with the complex formed in step a), or administering to the host organism, preferably through injection, the complex formed in step a).

Preferably, said host cell is a non human mammal cell or a human cell.

Preferably, the host organism is a human being or a non human mammal, although applying the hereabove method to other higher organisms such as plants cannot be excluded.

The invention also relates to a complex formed between a nucleic acid and a lipophilic composition or a lipid vesicle such as defined hereabove.

The invention further relates to a composition comprising a complex formed between a nucleic acid and a lipophilic composition or a lipid vesicle such as defined hereabove.

As previously already mentioned, the complexes formed between a nucleic acid of interest and a lipophilic compound or a lipid vesicle of the invention may be administered by any suitable method for introducing them into the cells of a human being or an animal, such as through injection into interstitial spaces of the tissues (heart, muscle, skin, lung, liver, intestines, and so on). Preferably, complexes come in the form of a composition also comprising a physiologically compatible vehicle.

In a particular embodiment of administration of the complexes according to the invention, the composition comprising these complexes presents in a form suitable for administration through an aerosol, for example for inhalation.

For injecting a complex between a lipophilic compound or a lipid vesicle of the invention and a nucleic acid of interest, the amount of DNA, RNA or DNA/RNA of interest for an injection dose does advantageously range from 0.005 mg/kg to 50 mg/kg of weight of the human being or animal to be treated. Preferably, the amount of nucleic acid does range from 0.005 mg/kg to 20 mg/kg, and most preferably from 0.05 mg/kg to 5 mg/kg.

Of course, the person skilled in the art is able to adapt the amount of nucleic acid in an injection dose, especially depending on the disease to be treated and of the injection site.

The amount of nucleic acid for an injection dose is determined by the person skilled in the art.

It is a further object of the present invention to provide a method for introducing in vivo a nucleic acid of interest into the cells of a host organism, said method comprising the following steps of:
a) contacting said nucleic acid with a lipophilic composition or with a lipid vesicle such as defined hereabove so as to obtain a complex formed between said nucleic acid, one the one hand, and said compound or said lipid vesicle, on the other hand;
b) administering the complexes formed at step a) to said host organism.

As already mentioned, the host organism is preferably a human being or a non human mammal, although it could also be a plant.

Generally speaking, the complexes formed between a nucleic acid of interest and a lipophilic composition or a lipid vesicle of the invention are present in a suitable liquid solution, such as sterile and pyrogen-free, distilled water, in suitable complex amounts. The solution may be used as such, or may further comprise one or more stabilizing agent(s), as Tween® (20, 40, 60 or 80), NaCl, or DMPE-PEG 5000.

The present invention further relates to a pharmaceutical composition comprising a complex formed between a nucleic acid of interest and a lipophilic composition or a lipid vesicle of the invention, if necessary in association with one or more physiologically compatible vehicle(s) or excipient(s).

As a rule, the compounds of formula (I) and the compounds of formula (XI) defined in the present specification form part of the invention.

Especially, the lipophilic compounds of formula (XI) are new and therefore also form part of the invention.

It is also therefore an object of the present invention to provide a lipophilic compound of following formula (XI):

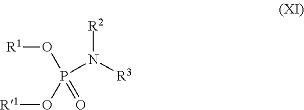

wherein:
(i) R¹ and R′¹ each represent independently from each other, an alkyl chain having from 10 to 24 carbon atoms, a monoalkenyl or a polyalkenyl chain having from 10 to 24 carbon atoms, the polyalkenyl chain having from 2 to 4 double bonds, or a monoalkynyl or a polyalkynyl chain having from 10 to 24 carbon atoms, the polyalkynyl chain having from 2 to 4 triple bonds;
(ii) R² is a hydrogen atom or an alkyl chain having from 1 to 4 carbon atoms;
(iii) R³ is a group of formula:

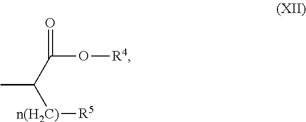

wherein
R⁴ is an alkyl group having from 1 to 4 carbon atoms;
n is an integer equal to 1, 2, 3 or 4; and
R⁵ is a group selected from:

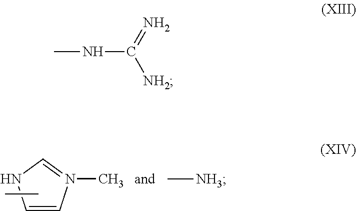

or

As a rule, the R¹, R′¹, R², R³, R⁴ and R⁵ groups have the same meaning as previously defined in the present specification.

In a first preferred embodiment, the R⁵ group has the following formula:

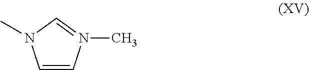

In a second preferred embodiment, the R¹ and R′¹ groups are the same and each represent the monoalkenyl chain C_{18:1}, wherein the first number is the number of carbon atoms in the alkenyl chain and the second number is the number of double bonds in the alkenyl chain.

It is a further object of the present invention to provide a lipophilic compound of following formula (I):

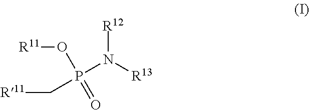

wherein (i) R¹¹ and R′¹¹ each represent independently from each other, an alkyl chain having from 10 to 24 carbon atoms, a monoalkenyl or a polyalkenyl chain having from 10 to 24 carbon atoms, the polyalkenyl chain having from 2 to 4 double bonds, or a monoalkynyl or a polyalkynyl chain having from 10 to 24 carbon atoms, the polyalkynyl chain having from 2 to 4 triple bonds;
(ii) R¹² is a hydrogen atom or an alkyl chain having from 1 to 4 carbon atoms;
(iii) R¹³ is a group of formula:

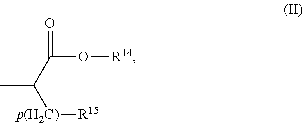

wherein
R¹⁴ is an alkyl group having from 1 to 4 carbon atoms;
p is an integer equal to 1, 2, 3 or 4; and
R¹⁵ is the following group:

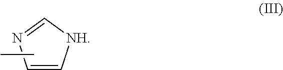

As a rule, the R¹¹, R′¹¹, R¹², R¹³, R¹⁴ and R¹⁵ groups have the same meaning as previously defined in the present specification.

In a first preferred embodiment, the R¹⁵ group is selected from the following groups:

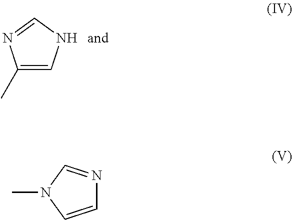

In a second preferred embodiment, the R¹⁶ group is selected from the following groups:

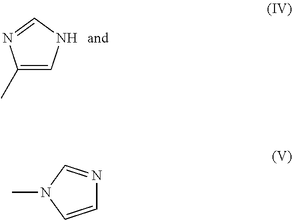

In a third preferred embodiment, the R¹¹ and R′¹¹ groups are selected from:
a tetradecyl group,
an oleyl group, and
C_{18:2} and C_{18:3} polyalkenyl groups, wherein the first number is the number of carbon atoms in the alkenyl chain and the second number is the number of double bonds in the alkenyl chain;
a C_{18:1} monoalkenyl group, wherein the first number is the number of carbon atoms in the alkenyl chain and the second number is the number of double bonds in the alkenyl chain.

In a fourth preferred embodiment, the R¹¹ and R′¹¹ groups are the same.

In compounds of formula (I) and in compounds of formula (XI), —NR¹²R¹³ and —NR²R³ portions, respectively, consist in amino acid derivatives, especially natural amino acid derivatives. Such structural characteristic of the compounds of formula (I) and (XI) of the invention could explain, at least partially, their reduced cytotoxic properties, when these amino acid derivatives are combined in the presence of a linkage group (“linker”) of the phosphoramidate type.

Another technical characteristic of compounds of formula (XI), wherein the R³ group is a group of formula (XII), is that the —OR⁴ ester group, which is not directly implied in the formation of complexes between these cationic, lipophilic compounds and the nucleic acid(s) of interest, may possess advantageous functions.

Firstly, the ester group —C(O)—OR⁴ acts as a hydrogen bond acceptor, which makes it possible to increase the interacting forces between the nucleic acid(s) of interest and the cationic, lipophilic compound of formula (XI), and therefore to improve the cohesion of the lipophilic compound-nucleic acid complexes.

Secondly, the —C(O)—OR⁴ ester group may be hydrolyzed after its internalization to the cell, especially through an acid-catalyzed hydrolysis reaction because of the acidic environment of the endosomes, or through an enzyme-mediated hydrolysis reaction because of the presence of esterases in the endosomes, being understood that the hydrolysis may be at the same time an acid hydrolysis for some cationic, lipophilic compounds and an enzymatic hydrolysis for other cationic, lipophilic compounds. After hydrolysis of the ester group, the lipophilic compound, that was initially a cationic compound, becomes zwitterionic, which may improve the intracellular routing of the lipophilic compound and thus may increase its ability to efficiently transfect human or animal host cells.

Similar observations may be made for neutral lipophilic compounds of formula (I), wherein the R¹³ group is a group of formula (II), since the ester group —C(O)—OR¹⁴ may be hydrolyzed in the same way as the ester group —C(O)—OR⁴ of compounds of formula (XI). In an acidic medium, or in the presence of esterases, compounds of formula (I) become anionic lipophilic compounds, which also may improve the transfection ability of a lipophilic composition of the invention.

Additional characteristics of the lipophilic compounds of formulas (I) and (XI) and of their preparation method, will be detailed hereunder.

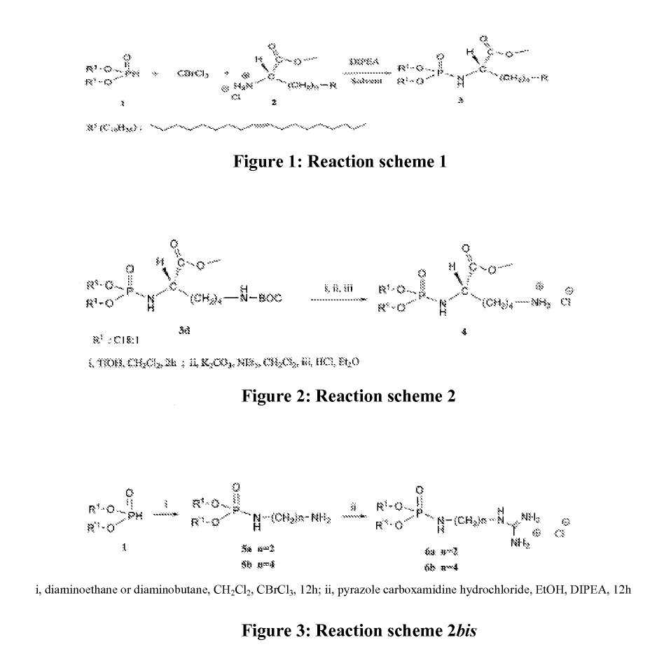

In scheme 1 (FIG. 1), DIPEA is diisopropyl ethylamine, and R (or R⁵) is the aminoester basic lateral moiety which, protonated at a physiological pH, enables the DNA complexation. To be mentioned as natural aminoacid-derived aminoesters are especially arginine and lysine methyl esters, (pKa=12.48 and 10.57, respectively). Homoarginine (pKa=12.5) is also to be mentioned, for its similarity to arginine. Because of the particular properties of histidine due to the side position of the imidazole ring, compounds comprising this aminoester have also been synthesized. This imidazole ring does possess a pKa of 6.04, which means that about 5% of the imidazole ring is protonated at a physiological pH, and that the remaining 95% will be able to act as a neutral co-lipid.

Summarized in table 1 in the example section are some embodiments of vectors that have been obtained by carrying out the synthesis scheme 1 illustrated on FIG. 1. Yields are given as an indication and have not been optimized.

For applying the same synthesis principle to the lysine methyl ester, it was necessary to use the protected aminoester, i.e. N-BOC lysine methylester. After phosphorylation, as described in scheme 1 (FIG. 1), the deprotection is carried out by use of trifluoroacetic acid, and thereafter the triflate anion is replaced by a chloride anion to obtain in fine phosphoramidate 4, as described in FIG. 2.

Other phosphoramidates of formulas (I) and (XI) have been synthesized, the R¹³ group of which is a group of formula —(CH₂)_{q}—R¹⁶ or the R³ group of which is a group of formula —(CH₂)_{q}—R⁶. FIG. 3 illustrates for example the synthesis principle of compounds 6 comprising a guanidine moiety, like arginine. It consists in preparing amine derivatives 5 by condensing diamines on a lipid phosphite, thereafter in guanidyling the amine through a guanydilation reactant as pyrazole-1-carboxamidine. The synthesis of the latter compounds is illustrated on FIG. 3. On FIG. 3, the references used have the following meanings: i) CBrCl₃, 2-diaminoethane or 1,4-diaminobutane, CH₂Cl₂., 20° C. ii) 1H-pyrazole-1-carboxamidine monohydrochloride, DIPEA, ethanol, 79° C.

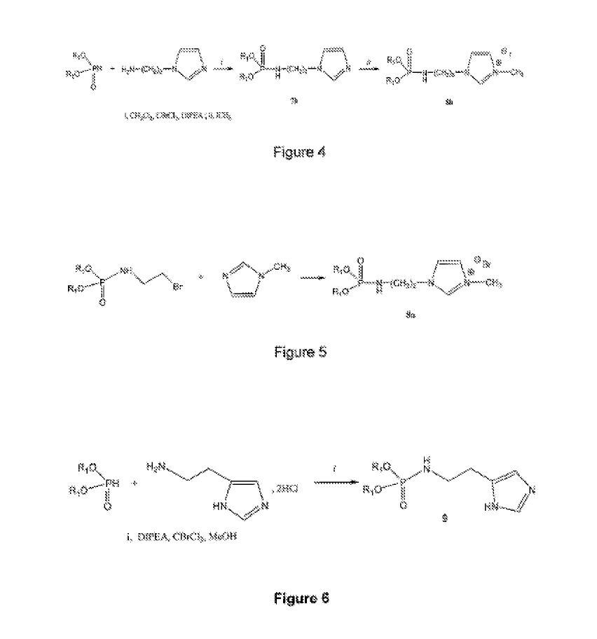

Phosphoramidates of formulas (I) and (XI) were also prepared, the R¹³ group of which is a group of formula —(CH₂)_{q}—R¹⁶ or the R³ group of which is a group of formula —(CH₂)_{q}—R⁶, which have an imidazole ring, like histidine, as illustrated on FIG. 4. FIG. 4 illustrates an example of how to obtain an imidazole ring-containing phosphoramidate. On FIG. 4, the references used have the following meanings: I i) CBrCl₃, DIPEA, 3-aminopropylimidazole, CH₂Cl₂, 20° C. ii) CH₃I, excess, 20° C., 16 h.

Since the imidazole pKa is about 6, this heterocycle is only partially protonated at a physiological pH. To make sure the cationic charge is constant, the corresponding imidazolium salts 8 were also synthesized. To this end, N-methyl imidazole has been condensed on a brominated phosphoramidate as illustrated on FIG. 5 (synthesis of 8a), or phosphoramidate 7b has been quaternized through the use of methyl iodide (synthesis of 8b), as illustrated on FIG. 4.

In these examples, the imidazole ring is bound to the rest of the molecule through one of the nitrogen atoms. A lipid 9 has also been synthesized, the imidazole ring of which is bound to the rest of the molecule through one of the carbon atoms in the ring. To this end, histamine was reacted on a lipid phosphite, as illustrated on FIG. 6. On FIG. 6, the references used have the following meanings: I i) CBrCl₃, DIPEA, MeOH, 20° C.

A new family of phosphoramidates has thus been synthesized and evaluated for the first time, which all have in common their lipid component part (oleic chains, that is to say with 18 carbon atoms and a central unsaturation) and a polar part derived from a natural amino-acid or from part of its components.

This family comprises therefore cationic lipids and for some of them their precursors, like compound 7b, which is a neutral lipid.

The present invention further relates to methods for preparing the lipophilic compounds of formulas (I) and (XI) defined in the present specification.

As a rule, compounds of formula (I) wherein the R¹³ group consists in a group of formula (II); as well as compounds of formula (XI), wherein the R³ group consists in a group of formula (XII), may be prepared according to scheme 1 of the method illustrated on FIG. 1.

In scheme 1 illustrated on FIG. 1:
for preparing compounds of formula (I):
   the R¹¹ and R′¹¹ groups do correspond to the two R¹ and R′¹ groups, respectively;
   the R¹⁴ group corresponds to the R⁴ group;
   the R¹⁵ group corresponds to the R⁵ group;
   the —(CH₂)ₚ-group corresponds to the —(CH₂)ₙ-group;
for preparing compounds of formula (XI):
   the R¹ and R′¹ groups do correspond to the two R¹ and R′¹ groups, respectively;
   the R⁴ group corresponds to the R⁴ group;
   the R⁵ group corresponds to the R⁵ group;
   the —(CH₂)ₙ-group corresponds to the —(CH₂)ₙ-group;

Compounds of formula (XI), wherein the R³ group consists in a group of formula —(CH₂)ₒ—R⁶, and more specifically those compounds wherein the R⁵ group consists in a group of formula (XIII), may be prepared according to scheme 2 of the method illustrated on FIG. 2.

Compounds of formula (XI), wherein the R³ group consists in a group of formula —(CH₂)ₒ—R⁶, and more specifically those compounds wherein the R⁵ group consists in a group of formula (XIV), may be prepared, from a compound of formula (I), according to scheme 4 of the method illustrated on FIG. 3, or according to scheme 5 illustrated on FIG. 4.

Compounds of formula (I) wherein the R¹³ group consists in a group of formula —(CH₂)ₒ—R¹⁶, and more specifically those compounds wherein the R¹⁵ group consists in a group of formula (IV), may be prepared according to scheme 3 of the method illustrated on FIG. 5.

Compounds of formula (I) wherein the R¹³ group consists in a group of formula —(CH₂)ₒ—R¹⁶, and more specifically those compounds wherein the R¹⁵ group consists in a group of formula (V), may be prepared according to scheme 6 of the method illustrated on FIG. 6.

The detailed synthesis procedures for compounds of formula (I) and compounds of formula (XI) are described in the examples.

As a rule, the cationic, lipophilic compounds of formula (XI) may present in the form of salts with an anion, that is to say in the form of a salt with all organic or mineral molecules which is negatively charged in a solution at a physiological pH. In a first aspect, the lipophilic compounds of formula (XI) may be prepared in the form of salts with an anion, where said anion may be chosen from CF₃CO₂⁻, CF₃SO₃⁻, HSO₄⁻ and a halogen. Said halogen may be chosen from Cl⁻, Br⁻ and I⁻. In a second aspect, when a compound of formula (XI) is added to a saline solution at physiological neutral pH, it forms a salt with the anions that are present in said saline solution. For example, in a cell culture medium, or in a body fluid from a human or an animal body, a cationic, lipophilic compound of formula (XI) is traditionally found in the form of a chloride salt.

The present invention will be now illustrated by means of the following examples, without being limited thereto.

### Examples

### Synthesis Procedure for Compounds of Formulas (I) and (XI)

### Example 1

### Synthesis of a Dioleoyl Phosphoramidate Compound Derived from Histidine Methyl Ester

2.91 g of dioleylphosphite (5 mmol), 920.35 mg of histidine methyl ester dihydrochloride (5 mmol), 15 mL of MeOH and 550 μL of CBrCl₃ (5.5 mmol) are combined together. The mixture is placed at a temperature lower than 5° C. in an ice/acetone bath. 2.6 mL of DIPEA (15 mmol) are then added thereto and the mixture is stirred at this temperature for one hour, then for one night at room temperature.

A purification through a silica gel column chromatography with for elution a mixture of CHCl₃/MeOH (90/10) enables the isolation of a pale yellow oil with a yield of 29% (m=1.08 g).

The structure of the compound is controlled with a proton, phosphorus-31 and carbon-13 NMR, and a mass spectrometry:

### ¹H-NMR in ppm (CDCl₃)

0.86 (t, 6H, CH₃, ³J_{H-H}=6.6 Hz); 1.26 (m, 44H, CH₂); 1.65 (m, 4H, CH₂ —O); 1.99 (m, 8H, CH₂ α-CH═CH); 3.08 (d, 2H, CH₂Im, ³J_{H-H}=5.3 Hz); 3.71 (s, 3H, OCH₃); 3.80 (t, NH, 10 Hz); 3.98 (m, 4H, CH₂ —O, ³J_{H-H}=³J_{P-H}=6.4 Hz); 4.12 (m, CH(NH)); 5.32 (m, 4H, CH═CH); 6.80 (s, H¹); 7.53 (s, H²)

### ¹³C-NMR in ppm (CDCl₃):

14.1 (s, CH₃); between 22.6 and 32.0 (plurality of singlets, CH₂); 30.3 (d, CH₂ —O, ³J_{P-C}=6.2 Hz); 30.9 (s, CH₂Im); 52.3 (s, OCH₃); 54.3 (s, CH(NH)); 66.1 (d, CH₂ —O, ²J_{P-C}=6.6 Hz); 115.3 (s, C¹); 129.6 (s, CH═CH); 129.7 (s, CH═CH); 131.5 (s, _{Quaternary}C); 134.8 (s, C²); 172.8 (s, CO)

### ³¹P-NMR in ppm (CDCl₃):

7.6 (s)

### Mass Spectrometry:

ESI for C₄₃H₈₁N₃O₅P, [M+H]⁺ calculated 750.59139, found 750.5905

### Example 2

### Synthesis of a Dioleoyl Phosphoramidate Compound Derived from Arginine Methyl Ester

2.80 g of dioleylphosphite (4.8 mmol), 1.27 g of arginine methyl ester dihydrochloride (4.8 mmol), 15 mL of MeOH and 550 μL of CBrCl₃ (5.5 mmol) are combined together. The mixture is placed at a temperature lower than 5° C. in an ice/acetone bath. 2.50 mL of DIPEA (14.4 mmol) are then added thereto and the mixture is stirred at this temperature for one hour, then for one night at room temperature.

A purification through a silica gel column chromatography with for elution a mixture of CHCl₃/MeOH (90/10) enables the isolation of a pale yellow oil with a yield of 31%.

The structure of the compound is controlled with a proton, phosphorus-31 and carbon-13 NMR, and a mass spectrometry:

### ¹H-NMR in ppm (DMSO):

0.83 (t, 6H, CH₃, ³J_{H-H}=6.6 Hz); 1.23 (m, 44H, CH₂); 1.53 (m, 4H, CH₂ —O); 1.96 (m, 8H, CH₂ α-CH═CH); 3.95 (m, 4H, CH₂ α-O, ³J_{H-H}=³J_{P-H}=6.4 Hz); 5.31 (m, 4H, CH═CH), 1.52 (m, 2H²); 1.65 (m, 2H¹); 3.10 (m, 2H³); 3.60 (m, CH); 3.61(s, OCH₃); 5.42 (t, NH¹); 7.80 (m, NH²)

### ¹³C-NMR in ppm (DMSO):

13.8 (s, CH₃); between 22.0 and 31.3 (plurality of singlets, CH₂); 25.1 (s, C²); 29.0 (d, CH₂ —O. ³J_{P-C}=6.2 Hz); 30.0 (s, C¹); 39.9 (s, C³); 51.6 (s, OCH₃); 53.6 (s, CH), 65.3 (d, CH₂ —O, ²J_{P-C}=6.6 Hz); 129.5 (s, CH═CH); 129.9 (s, CH═CH); 156.8 (s, C⁴); 173.5 (s, CO₂)

### ³¹P-NMR in ppm (CDCl₃):

8.8 (s)

### Mass Spectrometry:

ESI for C₄₃H₈₆N₄O₅P, [M+H]⁺, calculated 769.63359, found 769.6338

### Example 3

### Synthesis of a Dioleoyl Phosphoramidate Compound Derived from ‘Homoarginine Methyl Ester

2.80 g of dioleylphosphite (4.8 mmol), 1.34 g of homoarginine methylester dihydrochloride (4.8 mmol), 15 mL of MeOH and 550 μL of CBrCl₃ (5.5 mmol) are combined together. The mixture is placed at a temperature lower than 5° C. in an ice/acetone bath. 2.50 mL of DIPEA (14.4 mmol) are then added thereto and the mixture is stirred at this temperature for one hour, then for one night at room temperature.

A purification through a silica gel column chromatography with for elution a mixture of CHCl₃/MeOH (90/10) enables the isolation of a pale yellow oil with a yield of 30%.

The structure of the compound is controlled with a proton, phosphorus-31 and carbon-13 NMR, and a mass spectrometry:

### ¹H-NMR in ppm (DMSO):

0.83 (t, 6H, CH₃, ³J_{H-H}=6.6 Hz); 1.23 (m, 44H, CH₂); 1.45 (m, 2H³); 1.50 (m, 2H²); 1.53 (m, 4H, CH₂ —O); 1.65 (m, 2H¹); 1.96 (m, 8H, CH₂ α-CH═CH); 3.06 (m, 2H⁴); 3.54 (m, CH); 3.62 (s, OCH₃); 3.95 (m, 4H, CH₂ αO, ³J_{H-H}=³J_{P-H}=6.4 Hz); 5.31 (m, 4H, CH═CH); 5.38 (m, NH¹); 7.65 (m, NH²)

### ¹³C-NMR in ppm (DMSO):

13.8 (s, CH₃); between 22.0 and 31.3 (plurality of singlets, CH₂); 21.3 (s, C²); 27.8 (s, C³); 29.0 (d, CH₂ —O, ³J_{P-C}=6.2 Hz); 29.4 (s, C¹); 40.7 (s, C⁴); 51.4 (s, OCH₃); 54.5 (s, CH); 65.3 (d, CH₂ —O, ²J_{P-C}=6.6 Hz); 129.5 (s, CH═CH); 129.9 (s, CH═CH); 156.8 (s, C⁵); 173.5 (s, CO₂)

### ³¹P-NMR in ppm (DMSO):

8.9 (s)

### Mass Spectrometry:

ESI for C₄₄H₈₈N₄O₅P, [M+H]⁺, calculated 783.64924, found 783.6484

### Example 4

### Synthesis of a Dioleoyl Phosphoramidate Compound Derived from Lysine Methyl Ester

844 mg of dioleylphosphite (1.45 mmol), 430 mg of Boc-Lysine methylester hydrochloride (1.45 mmol), 15 mL of CHCl₃ and 200 μL of CBrCl₃ (2 mmol) are combined together. The mixture is placed at a temperature lower than 5° C. in an ice/acetone bath. 510 μL of DIPEA (2.90 mmol) are then added thereto and the mixture is stirred at this temperature for one hour, then for one night at room temperature.

After evaporation of the solvents, the mixture is taken up with ether and DIPEA precipitated salts are removed by filtration. (Yield 90%). 5 mL of CH₂Cl₂ and 5 mL of trifluoracetic acid are then added thereto and stirring is continued for two hours.

After evaporation of the solvents, the compound is solubilized in 10 mL of CH₂Cl₂, potassium carbonate in excess and one drop of Et₃N are added. Stirring is continued for three hours. After filtration, evaporation, addition of 10 mL of CH₂Cl₂ and of HCl in excess (in 2N ether solution), stirring is continued for thirty minutes.

A purification through a silica gel column chromatography with for elution a mixture of CHCl₃/MeOH (90/10) enables the isolation of a pale yellow oil with a yield of 70%.

The structure of the compound is controlled with a proton, phosphorus-31 and carbon-13 NMR, and a mass spectrometry:

### ¹H-NMR in ppm (DMSO):

0.83 (t, 6H, CH₃, ³J_{H-H}=6.6 Hz); 1.23 (m, 44H, CH₂); 1.35 (m, 2H²); 1.53 (m, 4H, CH₂ —O); 1.55 (m, 2H³); 1.60 (m, 2H¹); 1.96 (m, 8H, CH₂ —CH═CH); 2.70 (t, 2H⁴, ³J_{H-H}=7.4 Hz); 3.55 (m, CH); 3.71 (s, OCH₃); 3.95 (m, 4H, CH₂ α-O, ³J_{H-H}=³J_{P-H}=6.4 Hz); 5.31 (m, 4H, CH═CH); 5.35 (m, NH)

### ¹³C-NMR in ppm (DMSO):

13.8 (s, CH₃); between 22.0 and 31.3 (plurality of singlets, CH₂); 25.0 (s, C²); 26.5 (s, C³); 28.8 (s, C¹); 29.0 (d, CH₂ —O, ³J_{P-C}=6.2 Hz); 38.5 (s, C⁴); 51.6 (s, OCH₃); 53.9 (s, CH): 65.3 (d, CH₂ α-O, ²J_{P-C}=6.6 Hz); 129.5 (s, CH═CH); 129.9 (s, CH═CH fatty chains); 173.6 (s, CO₂)

### ³¹P-NMR in ppm (DMSO):

8.7 (s)

### Mass Spectrometry:

ESI for C₄₃H₈₆N₂O₅P, [M+H]⁺, calculated 741.62744, found 741.6262

### Example 5

### Synthesis of 3-propylmethylimidazolium dioleoyl phosphoramidate iodide

2.91 g of dioleylphosphite (5 mmol), 590 μL of aminopropyl imidazole (5 mmol), 15 mL of CH₂Cl₂ and 550 μL of CBrCl₃ (5.5 mmol) are combined together while maintaining the temperature at less than 5° C. in an ice/acetone bath. 960 μL of DIPEA (5.5 mmol) are then added thereto and the mixture is stirred at this temperature for one hour, thereafter for one hour at room temperature.

After evaporation of the solvents, the mixture is taken up with ether and DIPEA precipitated salts are removed by filtration.

After purification on silica gel (eluent CHCl₃/MeOH (90/10)), the phosphoramidate is obtained with a 82% yield as a pale yellow oil.

The structure of the intermediate compound is controlled with a proton, phosphorus-31 and carbon-13 NMR, thereafter quaternization is effected as follows:

2.8 g of the compound 5 (4 mmol) are solubilized in a large excess of ICH₃ (3 mL) and the mixture is stirred at room temperature for 16 hours. After evaporation, an orange oil is recovered. Yield is quantitative.

The structure of the compound is controlled with a proton, phosphorus-31 and carbon-13 NMR, and a mass spectrometry:

### ¹H-NMR in ppm (CD₃OD):

0.89 (t, 6H, CH₃, ³J_{H-H}=6.6 Hz); 1.28 (m, 44H, CH₂); 1.65 (m, 4H, CH₂ —O); 2.00 (m, 8H, CH₂ —CH═CH); 2.03 (m, 2H²); 2.94 (m, 2H¹); 3.67 (s, 3H, CH₃); 3.97 (m, 4H, CH₂ —O, ³J_{H-H}=³J_{P-H}=6.4 Hz); 4.30 (t, 2H³); 5.33 (m, 4H, CH═CH); 7.57 (s, CH²); 7.64 (s, CH³)

### ¹³C-NMR in ppm (CD₃OD):

14.5 (s, 2 CH₃); between 23.7 and 33.6 (plurality of singlets, CH₂); 26.7 (d, 2 CH₂ O, ³J_{P-C}=6.2 Hz); 28.3 (s, C²); 36.5 (s, CH₃); 38.5 (s, C¹); 48.0 (s, C³); 67.9 (d, 2 CH₂ α-O, ²J_{P-C}=6.6 Hz); 123.8 (s, C²); 125.1 (s, C³); 130.7 (s, CH═CH); 130.9 (s, CH═CH); 131.8 (s, C¹)

### ³¹P-NMR in ppm (CD₃OD):

9.9 (s)

### Mass Spectrometry:

ESI for C₄₃H₈₃N₃O₃P, [M+H]⁺, calculated 720.61721, found 720.6143

### Example 6

### Synthesis of a Dioleoyl Phosphoramidate Compound Derived from Histamine

2.91 g of dioleylphosphite (5 mmol), 920.3 mg of histamine dihydrochloride (5 mmol), 15 mL of MeOH and 550 μL of CBrCL₃ (5.5 mmol) are combined together. The mixture is placed at a temperature lower than 5° C. in an ice/acetone bath. 2.6 mL of DIPEA (15 mmol) are then added thereto and the mixture is stirred at this temperature for one hour, then for one night at room temperature.

A purification through a silica gel column chromatography with for elution a mixture of CHCl₃/MeOH (90/10) enables the isolation of a pale yellow oil with a yield of 30%.

The structure of the compound is controlled with a proton, phosphorus-31 and carbon-13 NMR, and a mass spectrometry:

### ¹H-NMR in ppm (CDCl₃):

0.86 (t, 6H, CH₃, ³J_{H-H}=6.6 Hz); 1.26 (m, 44H, CH₂); 1.65 (m, 4H, CH₂ —O); 1.99 (m, 8H, CH₂ —CH═CH); 2.79 (t, 2H, CH₂Im, ³J_{H-H}=5.5 Hz); 3.13 (m, NH) 3.19 (m, 2H, CH₂(NH)); 3.98 (m, 4H, CH₂ α-O, ³J_{H-H}=³J_{P-H}=6.4 Hz); 5.32 (m, 4H, CH═CH); 6.82 (s, H¹); 7.54 (s, H²)

### ¹³C-NMR in ppm (CDCl₃):

14.1 (s, CH₃); between 22.6 and 32.0 (plurality of singlets, CH₂); 30.3 (d, CH₂ —O, ³J_{P-C}=6.2 Hz); 29.1 (s, CH₂Im); 41.4 (s, CH₂(NH)); 66.1 (d, CH₂ α-O, ²J_{P-C}=6.6 Hz); 116.0 (s, C¹); 129.6 (s, CH═CH) 129.7 (s, CH═CH); 131.1 (s, _{Quaternary}C); 135.1 (s, C²)

### ³¹P-NMR in ppm (CDCl₃):

9.7 (s)

### Mass Spectrometry:

28: ESI for C₄₁H₇₉N₃O₃P, [M+H]⁺ calculated 692.58591, found 692.5854

### Example 7

### Synthesis of dioleoyl 2-ethylquanidinium phosphoramidate

In a first step, the synthesis of an aminophosphoramidate intermediate is effected as follows:

1.5 g of dioleylphosphite (2.6 mmol), 1.7 mL of diaminoethane (26 mmol), 10 mL of CH₂Cl₂ are combined together, then 260 μL of CBrCL₃ (2.6 mmol) are added dropwise thereto. Stirring is maintained all night long.

After washing with water, drying over magnesium sulfate and evaporation of the solvent, a yellow oil is recovered with a yield of 85%.

The structure of the compound is controlled with a proton, phosphorus-31 and carbon-13 NMR, and a mass spectrometry:

### ¹H-NMR in ppm (CDCl₃):

0.86 (t, 6H, CH₃, ³J_{H-H}=6.6 Hz); 1.26 (m, 44H, CH₂); 1.65 (m, 4H, CH₂ —O); 1.99 (m, 8H, CH₂ α-CH═CH); 2.64 (m, NH₂); 2.78 (t, 2H², ³J_{H-H}=5.5 Hz); 2.94 (m, 2H¹); 3.33 (m, NH); 3.98 (m, 4H, CH₂ α-O, ³J_{P-H}=6.4 Hz); 5.32 (m, 4H, CH═CH)

### ¹³C-NMR in ppm (CDCl₃):

14.1 (s, CH₃); between 22.6 and 32.0 (plurality of singlets, CH₂); 30.3 (d, CH₂ —O, ³J_{P-C}=6.2 Hz); 42.7 (s, C²); 43.7 (s, C¹); 66.1 (d, CH₂ α-O, ²J_{P-C}=6.6 Hz); 129.6 (s, CH═CH); 129.7 (s, CH═CH)

### ³¹P-NMR in ppm (CDCl₃):

10.0 (s)

### Mass Spectrometry:

ESI for C₃₈H₇₈N₂O₃P, [M+H]⁺, calculated 641.57501, found 641.5739

Guanidylation of the end amine is then effected as follows:

1.34 g of the previous intermediate compound, 309 mg of pyrazole carboxamidine hydrochloride (2.1 mmol), 10 mL of absolute ethanol and 367 μL of DIPEA (2.1 mmol) are combined together and heated to reflux for one night.

After evaporation of the solvent, a basic washing, an extraction with CH₂Cl₂, then a drying over MgSO₄ are carried out.

A purification through a silica gel column chromatography with for elution a mixture of CHCl₃/MeOH (90/10) enables the isolation of a pale yellow oil with a yield of 70%.

The structure of the compound is controlled with a proton, phosphorus-31 and carbon-13 NMR, and a mass spectrometry:

### ¹H-NMR in ppm (CD₃OD):

0.84 (t, 6H, CH₃, ³J_{H-H}=6.6 Hz); 1.24 (m, 44H, CH₂); 1.55 (m, 4H, CH₂ —O); 1.96 (m, 8H, CH₂ α-CH═CH); 2.85 (m, 2H¹); 3.14 (m, 2H²); 3.87 (m, 4H, CH₂ α-O, ³J_{H-H}=³J_{P-H}=6.4 Hz); 4.96 (m, NH); 5.32 (m, 4H, CH═CH); 7.54 (m, NH)

### ¹³C-NMR in ppm (CD₃OD):

14.1 (s, CH₃); between 22.6 and 32.0 (plurality of singlets, CH₂); 26.6 (d, CH₂ —O, ³J_{P-C}=6.2 Hz); 39.9 (s, C¹); 42.0 (s, C²); 65.4 (d, CH₂ α-O, ²J_{P-C}=6.6 Hz); 129.6 (s, CH═CH); 129.7 (s, CH═CH);

157.8 (s, C₄)

### ³¹P-NMR in ppm (CD₃OD):

9.9 (s)

### Mass Spectrometry:

ESI for C₃₉H₈₀N₄O₃P, [M+H]⁺, calculated 683.59681, found 683.5967

TABLE 1
Synthesis of the phosphoramidate aminoesters 3a-d.

| In- | | | | | | Product | Yield |
|---|---|---|---|---|---|---|---|
| put | Substrate | R | n | DIPEA | Solvent | Number | % |
| 1 | 2a (arginine methyl- ester) | 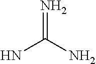 | 3 | 3 eq | MeOH | 3a | 31 |
| 2 | 2b (homo- arginine methyl ester) | 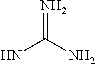 | 4 | 3 eq | MeOH | 3b | 30 |
| 3 | 2c (histidine methyl- ester) | 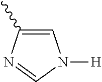 | 1 | 3 eq | MeOH | 3c | 29 |
| 4 | 2d | —NH—BOC | 4 | 2 eq | CHCl₃ | 3d | 90 |
| | (N-BOC | | | | | | |
| | lysine | | | | | | |
| | methyl- | | | | | | |
| | ester) | | | | | | |

### Example 2

### Evaluation of the Transfection Ability of the Lipophilic Compositions of the Invention

### A. Material and Methods

### A.1. Liposomes

A lipid film was prepared in a sterile flask under nitrogen by dry evaporating 1 mL of a 10.8 mM cationic lipid or a mixture of a cationic lipid/neutral lipid (molar ratio 1:1) in ethanol. The film was then hydrated with 1 mL of 10 mM HEPES buffer, pH 7.4, the solution was vigorously stirred for 3 min, then allowed to rest at 4° C. After 2 h, the solution was sonicated for 15 min in an ultrasonic bath at 37 kHz (Bioblock ultrasonic bath, Bioblock Scientific, Illkirch, France).

### A.2. DNA/Liposome Complexes

18 μl of a solution of liposomes at 5.4 mM were diluted in 200 μl of 10 mM HEPES buffer, pH 7.4. After 15 min, 7.5 μg of a plasmid encoding the gene of the luciferase (pTG11033; 9514 bp, Transgene S. A., Strasbourg, France) in 20 μl of 10 mM HEPES buffer, pH 7.4, were added to the liposomes and the mixture was allowed to rest for 30 min. The solution comprising the DNA/liposome complexes was adjusted to 1.5 ml with some culture medium free of serum and at 0.15M NaCl with a solution of 5M NaCl.

### A.3. Transfections

Two days before transfection, 293T7 cells (human kidney embryonal fibroblasts) were seeded with 1×10⁵ cells in 1 mL of a culture medium in a 24-well plate. The day of the transfection, cells achieved 80% confluence. Cells were washed twice with some medium free of serum prior to adding 2.5 μg of DNA in the form of DNA/liposome complexes. After 4 h of incubation at 37° C., the medium was removed and cells were cultured for 48 h with complete culture medium.

A.4. Measuring the Expression of the Luciferase in Transfected Cells (Procedure Adapted from De Wet (1987).

The culture medium of the cells was removed and each well was rinsed with 500 μL of PBS. Cell detachment was obtained with 500 μL of the PBS+trypsin per well for 5 min at 37° C. The cell suspension was then centrifuged for 5 min at 250×g (1500 rpm) at 20° C. 400 μL of lysis buffers (CCLR, Promega) were added on the cell pellet, which was allowed to incubate onto ice for 15 min. After Vortex-stirring, centrifugation was effected at 12 000×g for 2 min at 4° C. The luciferase activity contained in 20 μL of supernatant was measured using a luminometer (Bertold Lumat LB 9501) for 10 seconds after injection of 100 μL of luciferin (Luciferase Assay Reagent, Promega). The light transmitted was converted to arbitrary units relative to the protein amount in the sample determined through the bicinchoninic acid method.

### A.5. Protein Assay:

The protein amount in the lysate was evaluated through the BCA method (H. Hill and G. Straka, 1998). Proteins were contacted with bicinchoninic acid (BCA) and copper ions in an alkaline medium. The copper ions resulting from the protein-mediated reduction thereof formed stable, colored complexes with BCA. Coloration is objectified through spectrophotometric determination (562 nm). Once a standard range was obtained using bovine serum albumin, the weight of protein contained in the cell lysates was calculated.

### A.6. Transfection in the Damaged Achilles Tendon of a Rat.

The animal was narcotized in conformity with the legislation, then operated under a hood. After having carefully cleaned the leg with 70% ethanol, the skin was cut longitudinally by means of a curved scalpel blade size 12 (Swann Morton, Sheffield, England) from the top third of the gastrocnemius to the calcaneum (heel). The sheath surrounding the Achilles tendon was then cut. Cutting the tendon was effected by making a slight dent in the tendon along around 3 mm, longitudinally at the average lower third level.

Thereafter DNA was injected (formulated with the vectors or only naked) contained in a volume of 40 μl by means of a 22 Gauge-insulin syringe (Omnican, MWR). The wound was then sutured using a suture thread (Prolene, FS-2.19 mm, EThicon, Centravet). The leg was operated, then covered with Vétedine® (antiseptic/antifungal) (Vétoquinol, Centravet). The operated rats were then housed in a recovery cage.

### A.7. Measuring the Expression of Luciferase in the Transfected Tendons

Once the rats were euthanized with CO₂, they were promptly placed on ice. The tendon was quickly collected after incision and immerged into cold HBSS. Tendons were wiped, and then dipped into liquid nitrogen. Each tendon was then ground in a mortar with pestle. The thus obtained flakes were transferred to an Eppendorf tube comprising 500 μL of lysis buffer (CCLR, Promega). After a 3 hour-incubation on ice, centrifugation was conducted for 30 seconds at 13000×g. The supernatant was then transferred to a luminometer tube and RLU reading was performed as previously described. The light emission is expressed relative to the protein amount in the lysate.

### A.8. Tenocytes in Primary Culture

Tenocyte primary culture is prepared from Achilles tendon explants of “adult Wistar Han” rats weighting around 250 g. After euthanasia, the rats were immediately placed on ice. Under a laminar flow hood, the Achilles tendons were extracted using sterile surgical instruments after a carefully cleaning of the hind legs with alcohol. The tendons were immediately dipped into sterile, antibiotic-enriched HBSS (Hanks' Balanced Salt Solution) (penicillin 250 U/mL, streptomycin 250 μg/mL, kanamycin 100 μg/mL) and stored on ice. The tendons were rinsed 3 times repeatedly with antibiotic-enriched HBSS and contacted with 1 mL HBSS in a Petri dish. Using a sterile scalpel blade, the unwanted parts of the tendon were removed, such as muscle portions, fat or necrosed tissues. The desired parts were transferred to another Petri dish in the presence of HBSS and were dissected in small-sized explants of 0.3×0.5 mm side. The explants were recovered by means of a pipette and placed in 1 mL HBSS in a 15 ml-capacity Falcon tube. Centrifugation was performed at 250×g for 5 minutes. After having discarded the supernatant, the explants were recovered in complete medium (DMEM completed with 10% decomplemented fetal calf serum, vitamin C (44 μg/mL), of a mixture composed of penicillin/streptomycin (250 U/mL), kanamycin (91 μg/mL) (Sigma), gentamycin (91 μg/mL)). After 15 days, tenocytes did appear around the explants. Depending on the explant density, the tenocytes achieved confluence within the two to three following weeks. Cells were then detached using 2 mM EDTA-containing PBS and trypsin 2.5 μg/ml. Culture dishes were seeded with 100 000 cells per cm².

### B. Results

### B.1. Transfection in Human Cells

In the transfection tests of 293T line cells considered as a whole, it could be observed that the lipids 3a, 3b and 4 are more efficient when associated with the co-lipids 2c or 7b than with DOPE. In Table 2 hereunder, a few examples of these efficiency results are summarized, by indicating the observed luminescence values (expressed as “Total Relative Light Units (TRLU)” as mentioned in the procedure hereabove), the efficiency gain as compared to DOPE being put in brackets.

TABLE 2

Co-lipid

| Cationic lipid | — | DOPE | 2c | 7b |
|---|---|---|---|---|
| 3a | 7.10⁶ | 5.10⁶ | 5.10⁸ (100) | 10⁶ (0.2) |
| 3b | 9.10⁶ | 3.10⁶ | 3.10⁹ (1000) | 3.10⁷ (10) |
| 4 | 2.10⁷ | 3.10⁷ | 5.10⁷ (1.6) | 4.10⁷ (1.3) |

It could therefore be observed that except for the 3a/7b combination, there is an advantage to use co-lipids 2c and 7b rather than DOPE.

The person skilled in the art knows that results from in vitro biological assays do not always coincide with the results from in vivo assays. The applicants did therefore implement a series of in vivo assays to experiment the repair of damaged tendons of rats.

B.2. Transfection in Achilles Tendons with an Experimental Tendon Injury

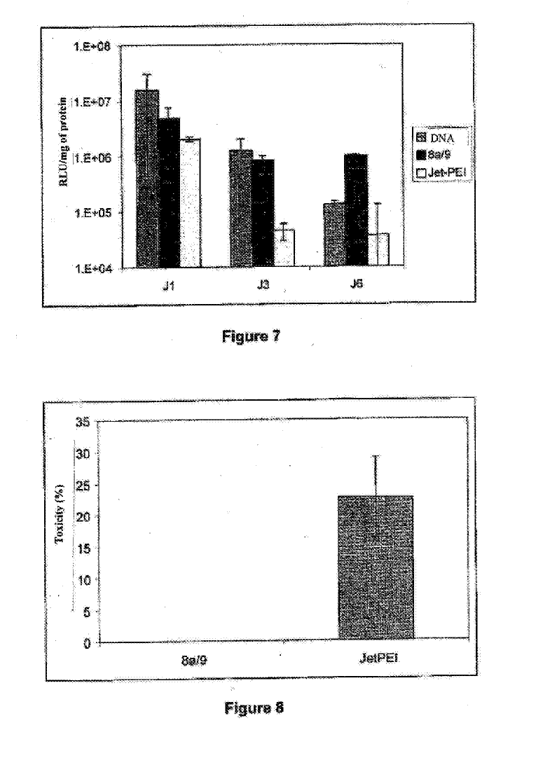

The results are given on FIG. 7.

The transfection efficiency after 24 h is high with 8a/9 with a level close to that obtained with naked DNA or DNA complexed with JetPEI. Vector 8a/9 is interesting because it makes it possible to express luciferase in a high amount (˜5 10⁶ RLU/mg) with a level which only decreases by a factor of 5 after day 3 and which is maintained up to day 6, whereas with naked DNA, it keeps decreasing. The gene expression obtained with JetPEI drastically decreases from the third day (100 times less). The results demonstrate that the cationic lipid 8a associated with the co-lipid 9 can be advantageously used for efficiently transfecting the Achilles tendon.

### Example 3

### Reduced Cytotoxicity of a Lipophilic Composition of the Invention

### A. Material and Methods

### Cytotoxicity Test:

Forty-height hours post transfection of the cells in vitro, their viability was tested. To this end, 50 μL of MTT (3-(4,5-dimethyl-2-thiazolyl)-2,5-diphenyl-2H-tetrazolium bromide) were added to 5 mg/mL per well of culture followed by a 4-hour incubation at 37° C. The medium of each well was recovered in Eppendorf tubes. Each well is then rinsed with PBS (twice 500 μL). Thereafter, 1 mL of acidified isopropanol and 200 μL of SDS (3%) were successively added to each well. After 30 min incubation at room temperature, the cell suspensions were recovered in 1.5 ml-capacity Eppendorf tubes and solubilized by vortexing. Lastly, 3×200 μL in each tube were collected and distributed on a 96-well plate, whereby absorbance was read at 570 nm.

### B. Results

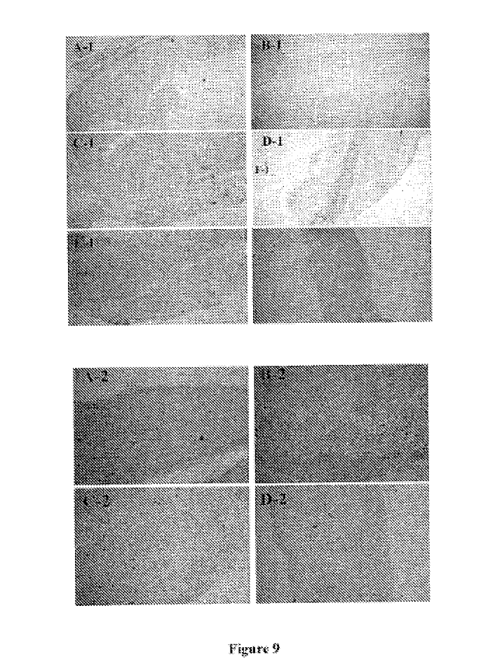

There was also substantially no cytotoxicity at all to be observed, as indicated on FIG. 8, wherein the combination 8a/9 was compared with a commercially available polymer (Jet PEI) which is also efficient but more cytotoxic.

### Example 4

### Repair of Rat Damaged Tendons Using a Lipophilic Composition of the Invention Complexed with Complementary DNA Encoding PDGF

### A. Material and Methods

It was reported that a treatment based on the recombinant protein PDGF induced a clear regeneration improvement of the rat damaged Achilles tendon. We did assess the effect of the PDGF-encoding gene transfer (PBlast45-hbFGF, InvivoGen) on the repair of the damaged tendon through the use of the liposomes 8a/9. To this end, histological analyses were made 3 or 6 days after the injury, followed with a treatment, or not. This plasmid does possess the gene encoding growth factor FGF-2 (bFGF) (basic human fibroblast growth factor) under the control of the constitutive promoter EF-1α-eIF4g (hybrid promoter of the human elongation factor EF-1α and of the 5′-untranslated region of the initiation factor eIF4g).

### B. Results

The results are given on FIG. 9.

Compared with the sections from undamaged tissues (FIGS. 9A-1, A-2 and 9B-1), the sections from the damaged tendons present degenerations (FIGS. 9C-1 and B-2); an improvement in the tissue aspect can be observed on the sections from the tissues treated on day 3 and more markedly on day 6 (FIGS. 9D-1, D-2 and 9F-1). By contrast, for the control tendons which were treated with a non relevant plasmid (pNF-CMV-luc), the tissue seemed to be more disorganized (FIGS. 9C-1, B-2 and E-1, C-2).

This vector suggests therefore the possibility to use efficiently a gene of therapeutic interest for repairing injuries to the tendon, or even against diseases associated with the tendon generally speaking. These are numerous and common diseases. They affect in the same manner sportsmen, who suffer from tendinopathies for 50% of the injuries caused by the sport, and the rest of the population.

1. A method for preparing a nucleic acid non viral vector composition, comprising:
combining a first lipophilic compound with second lipophilic compound of following formula (I): 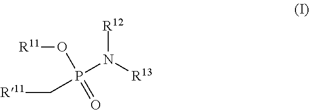
wherein (i) R¹¹ and R′¹¹ each represent independently from each other, an alkyl chain having from 10 to 24 carbon atoms, a monoalkenyl or a polyalkenyl chain having from 10 to 24 carbon atoms, the polyalkenyl chain having from 2 to 4 double bonds, or a monoalkynyl or a polyalkynyl chain having from 10 to 24 carbon atoms, the polyalkynyl chain having from 2 to 4 triple bonds;
(ii) R¹² is a hydrogen atom or an alkyl chain having from 1 to 4 carbon atoms;
(iii) R¹³ is selected from:
(iii-1) a group of formula 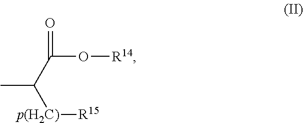
wherein
R¹⁴ is an alkyl group having from 1 to 4 carbon atoms;
p is an integer equal to 1, 2, 3 or 4; and
R¹⁵ is the following group: 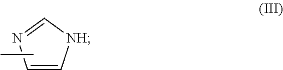
or
(iii-2) a group of formula —(CH₂)_{q}—R¹⁶, wherein
q is an integer equal to 1, 2, 3 or 4;
R¹⁶ is the following group:

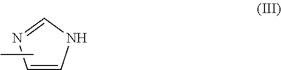

2. The method according to claim 1, characterized in that the first lipophilic compound is a cationic, lipophilic compound, that is able to form a complex with a nucleic acid.

3. A lipophilic composition comprising a combination of two lipophilic compounds, respectively:
a) a first cationic, lipophilic compound, that is able to form a complex with a nucleic acid; and
b) a second lipophilic compound of following formula (I): 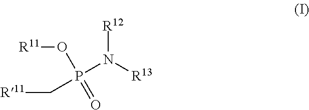
wherein (i) R¹¹ and R′¹¹ each represent independently from each other, an alkyl chain having from 10 to 24 carbon atoms, a monoalkenyl or a polyalkenyl chain having from 10 to 24 carbon atoms, the polyalkenyl chain having from 2 to 4 double bonds, or a monoalkynyl or a polyalkynyl chain having from 10 to 24 carbon atoms, the polyalkynyl chain having from 2 to 4 triple bonds;
(ii) R¹² is a hydrogen atom or an alkyl chain having from 1 to 4 carbon atoms;
(iii) R¹³ is selected from:
(iii-1) a group of formula 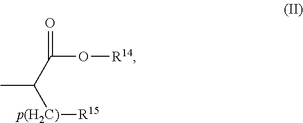
wherein
R¹⁴ is an alkyl group having from 1 to 4 carbon atoms;
p is an integer equal to 1, 2, 3 or 4; and
R¹⁵ is the following group: 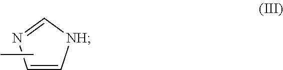
or
(iii-2) a group of formula —(CH₂)_{q}—R¹⁶, wherein
q is an integer equal to 1, 2, 3 or 4;
R¹⁶ is the following group:

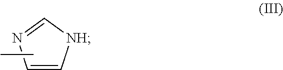

4. The composition according to claim 3, characterized in that in the second lipophilic compound, the R¹⁵ group is selected from the following groups:

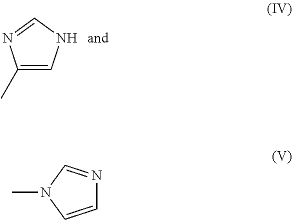

5. The composition according to claim 3, characterized in that in the second lipophilic compound, the R¹⁶ group is selected from the following groups:

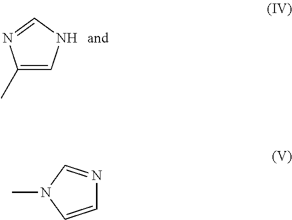

6. The composition according to claim 3, characterized in that in the compound of formula (I), the R¹¹ and R′¹¹ groups are selected from the group consisting of:
a tetradecyl group,
a phytanyl group,
an oleyl group,
the polyalkenyl C_{18:2} and C_{18:3} groups, wherein the first number is the number of carbon atoms in the alkenyl chain and the second number is the number of double bonds in the alkenyl chain, and
the C_{18:1} monoalkenyl group, wherein the first number is the number of carbon atoms in the alkenyl chain and the second number is the number of double bonds in the alkenyl chain.

7. The composition according to claim 6, characterized in that in the compound of formula (I), the R¹¹ and R′¹¹ groups are the same.

8. The composition according to claim 3, characterized in that the first cationic, lipophilic compound, that is able to form a complex with a nucleic acid, is selected from the group consisting of: 1,2-dioleyl-3 trimethylammonium deoxyglycerol (DOTAP), 1,2-dioleyl-3 trimethylammonium (DOTMA), dimethylammonium ethyloxycarbonyl cholesterol (DC-chol), dimethyldioctadecyl ammonium bromide (DDAB), 1,2- dimyristoyl-3 trimethylammonium deoxyglycerol, 1,2-dipalmitoyl-3 trimethylammonium deoxyglycerol, 1,2-dioleyl-3 trimethylammonium deoxyglycerol, 1,2-distearoyl-3 trimethylammonium deoxyglycerol, N-[1-[2,3-bis(oleoyloxy)]propyl-1]-N,N,N-trimethylammonium chloride, dioctadecyl amidoglycylspermine (DOGS), 2,3-dioleoyloxy-N-(2(sperminecarboxamido)-ethyl)-N,N-dimethyl-1-propanaminium trifluoroacetate (DOSPA), 1,2-dioleoyl-sn-glycero-3-ethyl phosphocholine (DOEPC), 1,2-dilauroyl-sn-glycero-3-ethyl phosphocholine, 1,2-dipalmitoyl-sn-glycero-3-ethyl phosphocholine, 1,2-distearoyl-sn-glycero-3-ethyl phosphocholine, 1,2-palmitoyl-oleoyl-sn-glycero-3-ethyl phosphocholine, 1-[2-(9(Z)-octadecenoyloxy)ethyl]-2-(8(Z)-heptadecenyl 1-3-(2-hydroxyethyl)imidazolium chloride .(DOTIM), 1-[2-tetradecanoyloxy)ethyl]-2-tridecyl-3-(2-hydroxyethyl)imidazolium chloride (DPTIM), 1-[2-tetradecanoyloxy)ethyl]-2-tridecyl-3-(2-hydroxyethyl)imidazolium chloride, 1,2-dioleoyl-3-dimethyl-hydroxyethyl ammonium bromide (DOR1); 1,2-dioleyloxypropyl-3-dimethyl-hydroxyethyl ammonium bromide (DORIE); 1,2-dioleyloxypropyl-3-dimethyl-hydroxypropyl ammonium bromide (DORIE-HP); 1,2-dioleyloxypropyl-3-dimethyl-hydroxybutyl ammonium bromide (DORIE-HB); 1,2-dioleyloxypropyl-3-dimethyl-hydroxypentyl ammonium bromide (DORIE-HPe); 1, 2-dimyristyloxypropyl-3-dimethyl-hydroxyethyl ammonium bromide (DMRIE); 1,2-dipalmityloxypropyl-3-dimethyl-hydroxyethyl ammonium bromide (DPRIE), 1,2-disteryloxypropyl-3-dimethyl-hydroxyethyl ammonium bromide (DSRIE).; L-histidine-(N,N-di-n-hexadecylamine)ethylamide (lipid 1); L-histidine-(N,N-di-n-hexadecylamine-N-methyl)ethylamide; L-histidine-cholesteryl-ethylamide, alanine-cholesteryl-ethylamide, and bis(guanidinium)-tren-cholesterol (BGTC).

9. The composition according to claim 3, characterized in that the first cationic, lipophilic compound, that is able to form a complex with a nucleic acid, is a lipophilic compound of following formula (XI): 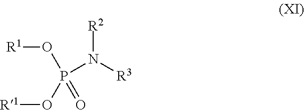
wherein:
(i) R¹ and R′¹ each represent independently from each other, an alkyl chain having from 10 to 24 carbon atoms, a monoalkenyl or a polyalkenyl chain having from 10 to 24 carbon atoms, the polyalkenyl chain having from 2 to 4 double bonds, or a monoalkynyl or a polyalkynyl chain having from 10 to 24 carbon atoms, the polyalkynyl chain having from 2 to 4 triple bonds;
(ii) R² is a hydrogen atom or an alkyl chain having from 1 to 4 carbon atoms;
(iii) R³ is selected from:
(iii-1) a group of formula 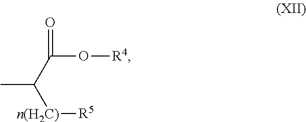
wherein
R⁴ is an alkyl group having from 1 to 4 carbon atoms;
n is an integer equal to 1, 2, 3 or 4; and
R⁵ is a group selected from: 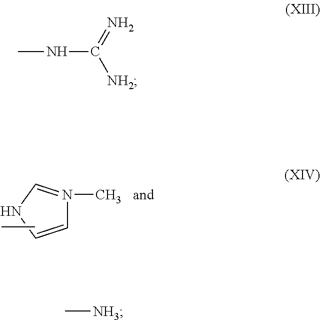
or
(iii-2) a group of formula —(CH₂)ₒ—R⁶, wherein
o is an integer equal to 1, 2, 3 or 4;
R⁶ is a group selected from:

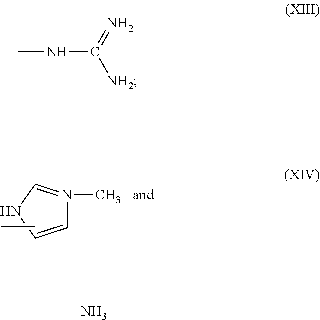

10. The composition according to claim 9, characterized in that in the first lipophilic compound, the R⁵ group is the following group:

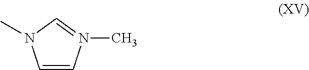

11. The composition according to claim 9, characterized in that in the first lipophilic compound, the R⁶ group is the following group:

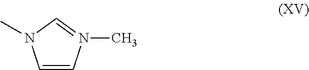

12. The composition according to claim 9, characterized in that in the compound of formula (XI), the R1 and R′1 groups are selected from:
a tetradecyl group,
an oleyl group,
a phytanyl group,
C_{18:2} and C_{18:3} polyalkenyl groups, wherein the first number is the number of carbon atoms in the alkenyl chain and the second number is the number of double bonds in the alkenyl chain; and
C_{18:1} monoalkenyl group, wherein the first number is the number of carbon atoms in the alkenyl chain and the second number is the number of double bonds in the alkenyl chain.

13. The composition according to claim 9, characterized in that in the compound of formula (XI), the R¹ and R′¹ groups are the same.

14. The composition according to claim 3, characterized in that the first lipophilic compound of formula (XI) is selected from the group consisting of:
arginine methyl ester dioleoyl-N-phosphoramidate,
homoarginine methyl ester dioleoyl-N-phosphoramidate,
lysine methyl ester dioleoyl-N-phosphoramidate,
2-ethylguanidinium dioleoyl-N-phosphoramidate,
4-butylguanidinium dioleoyl-N-phosphoramidate,
2-ethyl(N-methyl)imidazolium dioleoyl-N-phosphoramidate, and
3-propyl(N-methyl)imidazolium dioleoyl-N-phosphoramidate.

15. The composition according to claim 3, characterized in that the second lipophilic compound of formula (I) is selected from the group consisting of:
histidine methyl ester dioleoyl-N-phosphoramidate,
3-propylimidazole dioleoyl-N-phosphoramidate, and
histamine dioleoyl-N-phosphoramidate.

16. The composition according to claim 3, characterized in that it comprises the combination of the first lipophilic compound 8a and of the second lipophilic compound 9.

17. The composition according to claim 3, characterized in that it presents in the form of lipid vesicles.

18. A lipid vesicle substantially formed of a composition according to claim 3.

19. A lipid vesicle according to claim 18, consisting in an unilamellar vesicle.

20. A lipid vesicle according to claim 18, consisting in a multilamellar vesicle.

21. A method for introducing in vitro a nucleic acid into a host cell, characterized in that it comprises the following steps of:
a) contacting said nucleic acid with a composition according to claim 3 so as to obtain a complex between said nucleic acid, on the one hand, and said composition, on the other hand; and
b) incubating the host cell with the complex formed in step a).

22. A method for introducing in vitro a nucleic acid (DNA or RNA) into a host cell, characterized in that it comprises the following steps of:
a) contacting said nucleic acid with lipid vesicles according to claim 18 so as to obtain a complex between said nucleic acid, on the one hand, and said lipid vesicles, on the other hand; and
b) incubating the host cell with the complex formed in step a).

23. The method according to claim 22, characterized in that said host cell is a non human mammal cell or a human cell.

24. A complex formed between a nucleic acid and a composition according to claim 3.

25. A composition comprising a complex according to claim 24.

26. A pharmaceutical composition comprising a complex according to claim 24.

27. A lipophilic compound of following formula (XI): 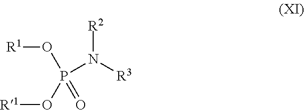
wherein:
(i) R¹ and R′¹ each represent independently from each other, an alkyl chain having from 10 to 24 carbon atoms, a monoalkenyl or a polyalkenyl chain having from 10 to 24 carbon atoms, the polyalkenyl chain having from 2 to 4 double bonds, or a monoalkynyl or a polyalkynyl chain having from 10 to 24 carbon atoms, the polyalkynyl chain having from 2 to 4 triple bonds;
(ii) R² is a hydrogen atom or an alkyl chain having from 1 to 4 carbon atoms;
(iii) R³ is a group of formula 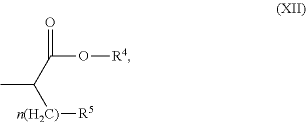
wherein
R⁴ is an alkyl group having from 1 to 4 carbon atoms;
n is an integer equal to 1, 2, 3 or 4; and
R⁵ is a group selected from: 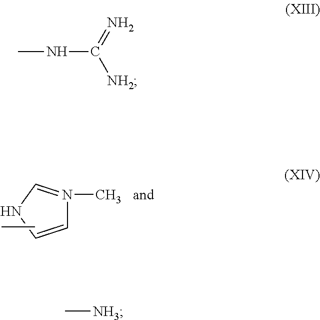
or

28. The lipophilic compound according to claim 27, characterized in that the R⁵ group has the following formula:

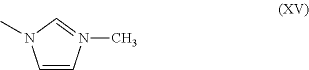

29. The lipophilic compound according to claim 27, wherein the R¹ and R′¹ groups are the same and each represent C_{18:1} monoalkenyl chain, wherein the first number is the number of carbon atoms in the alkenyl chain and the second number is the number of double bonds in the alkenyl chain.

30. A lipophilic compound of following formula (I): 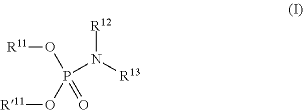
wherein
(i) R¹¹ and R′¹¹ each represent independently from each other, an alkyl chain having from 10 to 24 carbon atoms, a monoalkenyl or a polyalkenyl chain having from 10 to 24 carbon atoms, the polyalkenyl chain having from 2 to 4 double bonds, or a monoalkynyl or a polyalkynyl chain having from 10 to 24 carbon atoms, the polyalkynyl chain having from 2 to 4 triple bonds;
(ii) R¹² is a hydrogen atom or an alkyl chain having from 1 to 4 carbon atoms;
(iii) R¹³ is a group of formula 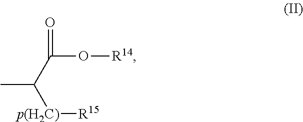
wherein
R¹⁴ is an alkyl group having from 1 to 4 carbon atoms;
p is an integer equal to 1, 2, 3 or 4; and
R¹⁵ is the following group:

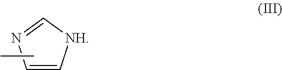

31. The lipophilic compound according to claim 30, characterized in that the R¹⁵ group is selected from the following groups:

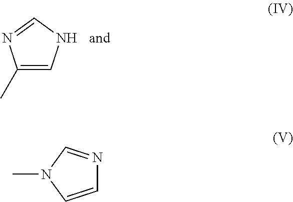

32. The lipophilic compound according to claim 30, characterized in that the R¹⁶ group is selected from the following groups:

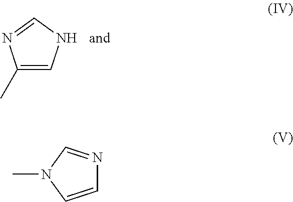

33. The lipophilic compound according to claim 30, characterized in that the R¹¹ and R′¹¹ groups are selected from:
a tetradecyl group,
an oleyl group, and
C_{18:2} and C_{18:3} polyalkenyl groups, wherein the first number is the number of carbon atoms in the alkenyl chain and the second number is the number of double bonds in the alkenyl chain;
C_{18:1} monoalkenyl group, wherein the first number is the number of carbon atoms in the alkenyl chain and the second number is the number of double bonds in the alkenyl chain.

34. The lipophilic compound according to claim 30, characterized in that the R¹¹ and R′¹¹ groups are the same.

35. A complex formed between a nucleic acid and lipid vesicles according to claim 18.

36. A composition comprising a complex according to claim 35.

37. A pharmaceutical composition comprising a complex according to claim 36.
